(19) United States
(12) Patent Application Publication
(10) Pub. No.: US 2026/0109772 A1
(43) Pub. Date: Apr. 23, 2026

(54) Methods for Reducing Alloantibody Levels in Subjects in Need of Solid Organ Transplantation

(71) Applicant: Regeneron Pharmaceuticals, Inc., Tarrytown, NY (US)

(72) Inventors: Nikhil SINGE, Fairfield, CT (US); Thomas D. NORTON, Wyckoff, NJ (US); Olivier HARARI, New York, NY (US); Andre LIMNANDER, New York, NY (US); Matthew SLEEMAN, Yorktown Heights, NY (US); Lorah PERLEE, Wilton, CT (US); Stephen M. CARPENTER, Solon, OH (US)

(21) Appl. No.: 19/116,305

(22) PCT Filed: Oct. 9, 2023
(86) PCT No.: PCT/US2023/034721

§ 371 Date: Mar. 27, 2025

Related U.S. Application Data
(60) Provisional application No. 63/414,705, filed on Oct. 10, 2022.
(60) Provisional application No. 63/539,027, filed on Sep. 18, 2023.

(51) Int. Cl. C07K 16/28; A61K 39/00; A61P 37/06

(52) U.S. Cl. CPC C07K 16/2878; A61P 37/06; C07K 16/2809; A61K 2039/505; C07K 2317/31

(57) ABSTRACT

B-cell maturation antigen (BCMA) is expressed on plasma cells. The present invention provides methods for desensitizing patients in need of a solid organ transplant to anti-HLA antibodies using bispecific antibodies that bind to both BCMA and CD3 and activate T cells via the CD3 complex in the presence of BCMA-expressing plasma cells. In certain embodiments, the bispecific antibodies of the present invention reduce alloantibody levels and/or reduce calculated panel reactive antibody levels to facilitate solid organ transplantation and/or improve long term graft survival and/or function.

### REFERENCE TO A SEQUENCE LISTING

This application incorporates by reference a computer readable Sequence Listing in ST.26 XML format, titled 11294WO01_Sequence, created on Oct. 8, 2023, and containing 48,180 bytes.

### FIELD OF THE INVENTION

The present invention pertains to the field of medicine, and relates to bispecific antibodies (and antigen-binding fragments thereof) that bind BCMA and CD3, and methods of use thereof for, e.g., desensitizing patients to anti-HLA alloantibodies prior to solid organ transplantation.

### BACKGROUND

B-cell maturation antigen (BCMA), also known as TNFRSF17, or CD269, is a type Ill transmembrane protein lacking a signal peptide and containing a cysteine-rich extracellular domain. BCMA, along with closely related proteins, promotes B-cell survival at distinct stages of development. BCMA is expressed exclusively in B-cell lineage cells, particularly in the interfollicular region of the germinal center as well as on plasmablasts and differentiated plasma cells. BCMA is selectively induced during plasma cell differentiation, and is required for optimal survival of long-lived plasma cells in the bone marrow.

CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a target antigen have been proposed for therapeutic uses involving targeting T cell immune responses to tissues and cells expressing the target antigen.

Kidney transplantation is the preferred treatment for end-stage kidney disease. Hemodialysis, although life-saving in the short-term, confers 1-year and 5-year mortality rates as high as 25% and 65%, respectively, whereas the 5-year mortality rate for patients treated with kidney transplantation is as low as 3%. However, for patients with high levels of preformed anti-human leukocyte antigen (HLA) antibodies, organ transplantation is infrequently performed due to an increased risk of antibody-mediated rejection (AMR) and shortened graft survival. About 30% of the approximately 95,000 patients on the kidney transplant waiting list in the United States are sensitized to HLA from prior transplants, blood transfusions, and pregnancies. About half of those (approximately 13,000 patients) are considered highly sensitized (i.e., expected to react to ≥80% of donor HLA types). The calculated panel-reactive antibody (cPRA) score represents the probability of encountering an incompatible donor for organ transplant candidates and is used as a measurement of sensitization level. While the kidney allocation system (KAS) has improved transplantation rates for highly sensitized individuals overall, 30% to 50% of patients with a cPRA score ≥90% remain on the kidney transplant waitlist for over 5 years, highlighting the unmet need for this subset of sensitized patients. Of the approximately 11,000 chronic kidney disease (CKD) patients with a cPRA 90% who are on the kidney transplant waitlist in the US, the majority (approximately 7,000) have cPRA scores ≥99%. These patients have a particularly high unmet need as evidenced by their more frequent removal from the waitlist, and death, due to medical comorbidities. Furthermore, for the approximately 2,000 patients on the kidney transplant waitlist with a cPRA≥99.9%, the average rate of transplantation is markedly lower than for other HLA-sensitized patients, despite the implementation of the KAS in 2014. Desensitization strategies that facilitate successful transplantation for these patients are therefore a high priority.

Transplant options for individuals sensitized to multiple or common HLAs are limited due to the risk of AMR and subsequent graft loss if transplanted with an HLA-incompatible kidney. The pathogenesis of AMR involves antibodies directed at HLA that are produced by plasma cells and possibly B cells. Other effector molecules, including cytokines and complement, have also been shown to contribute to kidney pathology. Antibody-mediated rejection can manifest as hyperacute rejection, which occurs within minutes after vascular anastomosis, often resulting in graft loss within hours. More commonly, AMR manifests in acute/active and chronic forms that develop over months or years. While hyperacute rejection is rare due to improved HLA screening and crossmatching, acute/active AMR rates are reportedly as high as 40% to 45% in HLA-sensitized patients despite the use of plasmapheresis and intravenous immune globulin (IVIG) desensitization techniques. While acute AMR can be managed with short-term immunosuppression in many cases, it is a strong risk factor for the development of chronic AMR, which is the most common cause of graft loss in the US. The Deterioration in Kidney Allograft Function (DeKAF) study showed that most patients with graft loss had evidence of chronic AMR, including deposition of the split C4 complement component on biopsy (Matas et al., Am J Transplant, 18(5):1140-1150, 2018). Furthermore, the risk of chronic AMR is increased 4-fold in prior recipients of a transplant that are highly sensitized to HLA, even after waiting for an HLA-compatible kidney (Schinstock et al., Transplantation, 101(10):2429-2439, 2017). Sensitization and the presence of anti-HLA alloantibodies is also implicated in delayed transplantation of other solid organs, including heart (Kransdorf et al., Transplantation, 101(9):1971-1976, 2017) and lung (Barac et al., An Thorac Surg., 110(2):414-423, 2020) transplantation. Hence, there remains a high unmet need for more effective therapies to facilitate transplantation, and to prevent post-transplant acute and chronic AMR in individuals highly sensitized to HLA.

### SUMMARY OF THE INVENTION

The present disclosure is generally directed to methods of using anti-BCMA x anti-CD3 bispecific antibodies (e.g., REGN5459 or REGN5458) to safely deplete B cell maturation antigen (BCMA)-expressing plasma cells and decrease anti-HLA alloantibodies to facilitate solid organ transplantation (e.g., kidney transplantation). Plasma cell-targeting therapies (as described herein), by eliminating the source of anti-HLA alloantibodies, will have a longer-lasting HLA desensitization effect, facilitating transplantation and reducing the risk of post-transplant AMR. Anti-BCMA x CD3 antibodies (e.g., REGN5459 and REGN5458) target the surface protein BCMA that is selectively expressed on plasma cells. The bispecific antibodies deplete plasma cells and newly activated BCMA-expressing B cells, including those that produce anti-HLA antibodies, without impairing regulatory T cell responses that promote tolerance after a transplant. The restricted pattern of BCMA expression in tissues makes it an attractive therapeutic target for prevention of AMR.

In one aspect, the present invention provides a method of reducing an alloantibody level in a subject in need of a solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell.

In some embodiments, reducing the alloantibody level in the subject comprises reducing the alloantibody level below a baseline alloantibody level measured prior to administration of the bispecific antibody or antigen-binding fragment thereof. In some cases, the method further comprises measuring a baseline alloantibody level in the subject prior to administration of the bispecific antibody or antigen-binding fragment thereof. In some cases, the alloantibody level and/or the baseline alloantibody level is measured by a single antigen bead assay, and the alloantibody level and/or the baseline alloantibody level corresponds to a peak immunodominant anti-HLA antibody mean fluorescence intensity. In some cases, the reduction in the alloantibody level corresponds to a reduction in a peak immunodominant anti-HLA antibody mean fluorescence intensity by ≥50% relative to the baseline alloantibody level.

In some embodiments, the alloantibody level is measured by a single antigen bead assay, and a reduction in the alloantibody level corresponds to a reduction in a peak immunodominant anti-HLA antibody mean fluorescence intensity to <5000.

In one aspect, the present invention provides a method of reducing a calculated panel reactive antibody (cPRA) level of a subject in need of a solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell.

In some embodiments, reducing the cPRA level of the subject comprises reducing the cPRA level of the subject below a baseline cPRA level determined prior to administration of the bispecific antibody or antigen-binding fragment thereof. In some cases, the cPRA level of the subject is reduced to: <99%; <98%; <97%; <96%; <95%; <94%; <93%; <92%; <91%; <90%; <89%; <88%; <87%; <86%; <85%; <84%; <83%; <82%; <81%; or <80%.

In one aspect, the present invention provides a method of reducing sensitization of a subject to anti-HLA antibodies prior to solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell, wherein the subject's risk of rejection at the time of organ transplant is no greater than that of a control population with a pre-transplant calculated panel reactive antibody (cPRA) level of 90%.

In some embodiments, the subject's risk of rejection at the time of organ transplant is no greater than that of a control population with a pre-transplant cPRA level of 80%. In some embodiments, the subject's risk of rejection at the time of organ transplant is no greater than that of a control population with a pre-transplant cPRA level of from 50% to <80%.

In one aspect, the present invention provides a method of reducing risk of allograft rejection in a subject following a solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell, wherein the subject's risk of rejection during a post-transplant interval is no greater than that of a control population with a pre-transplant calculated panel reactive antibody (cPRA) level of 90%.

In some embodiments, the subject's risk of rejection during the post-transplant interval is no greater than that of a control population with a pre-transplant cPRA level of 80%. In some embodiments, the subject's risk of rejection during the post-transplant interval is no greater than that of a control population with a pre-transplant cPRA level of from 50% to <80%. In some cases, the post-transplant interval is a 3-month interval beginning one day after organ transplant. In some cases, the post-transplant interval is a 6-month interval beginning one day after organ transplant. In some cases, the post-transplant interval is a 12-month interval beginning one day after organ transplant.

In some embodiments, graft function is maintained in the subject during the post-transplant interval at a function level equal to or greater than that of the control population.

In any of the various embodiments of the methods discussed above or herein, the subject may be a human.

In any of the various embodiments of the methods discussed above or herein, the solid organ may be selected from the group consisting of a kidney, a lung, a pancreas, or a heart. In some cases, the solid organ is a kidney. In some cases, the solid organ is a kidney, and the subject has been on the kidney transplant waitlist for 5 years or longer. In some cases, the subject suffers from chronic kidney disease. In some cases, the subject is on hemodialysis. In some cases, the subject is highly sensitized with end-stage renal failure requiring hemodialysis.

In any of the various embodiments of the methods discussed above or herein, the bispecific antibody or antigen-binding fragment thereof may be as discussed hereinbelow.

In some embodiments, the first antigen-binding domain comprises: (a) three heavy chain complementarity determining regions (HCDR1, HCDR2 and HCDR3) contained within a heavy chain variable region (HCVR) comprising the amino acid sequence of SEQ ID NO: 1; and (b) three light chain complementarity determining regions (LCDR1, LCDR2 and LCDR3) contained within a light chain variable region (LCVR) comprising the amino acid sequence of SEQ ID NO: 5. In some cases, the first antigen-binding domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4. In some cases, the first antigen-binding domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8. In some cases, the first antigen-binding domain comprises a HCVR comprising the amino acid sequence of SEQ ID NO: 1, and a LCVR comprising the amino acid sequence of SEQ ID NO: 5.

In some embodiments, the second antigen-binding domain comprises: (a) three heavy chain complementarity determining regions (HCDR1, HCDR2 and HCDR3) contained within a heavy chain variable region (HCVR) comprising the amino acid sequence of SEQ ID NO: 9 or SEQ ID NO: 13; and (b) three light chain complementarity determining regions (LCDR1, LCDR2 and LCDR3) contained within a light chain variable region (LCVR) comprising the amino acid sequence of SEQ ID NO: 5. In some cases, the second antigen-binding domain comprises: (a) a HCDR1 comprising the amino acid sequence of SEQ ID NO: 10 or SEQ ID NO: 14; (b) a HCDR2 comprising the amino acid sequence of SEQ ID NO: 11 or SEQ ID NO: 15; and (c) a HCDR3 comprising the amino acid sequence of SEQ ID NO: 12 or SEQ ID NO: 16. In some cases, the second antigen-binding domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8. In some cases, the second antigen-binding domain comprises: (a) HCDR1, HCDR2, HCDR3 domains, respectively, comprising the amino acid sequences of SEQ ID NOs: 10, 11, 12; and LCDR1, LCDR2, LCDR3 domains, respectively, comprising the amino acid sequences of SEQ ID NOs: 6, 7, 8; or (b) HCDR1, HCDR2, HCDR3 domains, respectively, comprising the amino acid sequences of SEQ ID NOs: 14, 15, 16; and LCDR1, LCDR2, LCDR3 domains, respectively, comprising the amino acid sequences of SEQ ID NOs: 6, 7, 8. In some cases, the second antigen-binding domain comprises: (a) a HCVR comprising the amino acid sequence of SEQ ID NO: 9, and a LCVR comprising the amino acid sequence of SEQ ID NO: 5; or (b) a HCVR comprising the amino acid sequence of SEQ ID NO: 13, and a LCVR comprising the amino acid sequence of SEQ ID NO: 5.

In some embodiments, (a) the first antigen-binding domain comprises HCDR1, HCDR2, HCDR3 domains, respectively, consisting of the amino acid sequences of SEQ ID NOs: 2, 3, 4, and LCDR1, LCDR2, LCDR3 domains, respectively, consisting of the amino acid sequences of SEQ ID NOs: 6, 7, 8; and (b) the second antigen binding domain comprises HCDR1, HCDR2, HCDR3 domains, respectively, consisting of the amino acid sequences of SEQ ID NOs: 10, 11, 12, and LCDR1, LCDR2, LCDR3 domains, respectively, consisting of the amino acid sequences of SEQ ID NOs: 6, 7, 8. In some cases, (a) the first antigen binding domain comprises a HCVR consisting of the amino acid sequence of SEQ ID NO: 1, and a LCVR consisting of the amino acid sequence of SEQ ID NO: 5; and (b) the second antigen binding domain comprises a HCVR consisting of the amino acid sequence of SEQ ID NO: 9, and a LCVR consisting of the amino acid sequence of SEQ ID NO: 5.

In some embodiments, (a) the first antigen-binding domain comprises HCDR1, HCDR2, HCDR3 domains, respectively, consisting of the amino acid sequences of SEQ ID NOs: 2, 3, 4, and LCDR1,LCDR2,LCDR3 domains, respectively, consisting of the amino acid sequences of SEQ ID NOs: 6, 7, 8; and (b) the second antigen binding domain comprises HCDR1,HCDR2,HCDR3 domains, respectively, consisting of the amino acid sequences of SEQ ID NOs: 14, 15, 16, and LCDR1,LCDR2,LCDR3 domains, respectively, consisting of the amino acid sequences of SEQ ID NOs: 6, 7, 8. In some cases, (a) the first antigen binding domain comprises a HCVR consisting of the amino acid sequence of SEQ ID NO: 1, and a LCVR consisting of the amino acid sequence of SEQ ID NO: 5; and (b) the second antigen binding domain comprises a HCVR consisting of the amino acid sequence of SEQ ID NO: 13, and a LCVR consisting of the amino acid sequence of SEQ ID NO: 5.

In some embodiments, the bispecific antibody or antigen-binding fragment thereof is a bispecific antibody comprising a human IgG heavy chain constant region. In some cases, the bispecific antibody comprises a heavy chain comprising a constant region comprising the amino acid sequence of SEQ ID NO: 33. In some cases, the bispecific antibody comprises a heavy chain comprising a constant region comprising the amino acid sequence of SEQ ID NO: 34. In some cases, the human IgG heavy chain constant region is isotype IgG1. In some cases, the human IgG heavy chain constant region is isotype IgG4. In some embodiments, the bispecific antibody comprises a chimeric hinge that reduces Fcγ receptor binding relative to a wild-type hinge of the same isotype.

In some embodiments, the bispecific antibody or antigen-binding fragment thereof is a bispecific antibody comprising a first heavy chain comprising the amino acid sequence of SEQ ID NO: 29, a second heavy chain comprising the amino acid sequence of SEQ ID NO: 30, and a common light chain comprising the amino acid sequence of SEQ ID NO: 32.

In some embodiments, the bispecific antibody or antigen-binding fragment thereof is a bispecific antibody comprising a first heavy chain comprising the amino acid sequence of SEQ ID NO: 29, a second heavy chain comprising the amino acid sequence of SEQ ID NO: 31, and a common light chain comprising the amino acid sequence of SEQ ID NO: 32.

In any of the various embodiments of the methods discussed above or herein, the bispecific antibody is administered in a dosing regimen comprising a split initial dose. In some embodiments, the bispecific antibody is administered to the subject at a dose of from 0.05 mg to 150 mg weekly.

In one aspect, the present invention provides a dosing regimen for use in any one of the embodiments of the methods discussed above or herein, wherein the dosing regimen comprises administration of a bispecific antibody to the subject at an initial dose during week one of the dosing regimen, at a secondary dose during week two of the dosing regimen, and at a tertiary dose during week three of the dosing regimen, wherein the tertiary dose is equal to or greater than the secondary dose, and the secondary dose is greater than the initial dose.

In some embodiments, the initial dose is from 0.05 mg to 5 mg. In some embodiments, the secondary dose is from 0.15 mg to 25 mg. In some embodiments, the tertiary dose is from 0.5 mg to 150 mg. In some cases, the initial dose is 0.05 mg, 0.15 mg, 0.5 mg, 1 mg, 1.5 mg or 5 mg. In some cases, the secondary dose is 0.15 mg, 0.5 mg, 1.5 mg, 3 mg, 5 mg, 15 mg or 25 mg. In some cases, the tertiary dose is 0.5 mg, 1.5 mg, 5 mg, 10 mg, 15 mg, 20 mg, 40 mg, 50 mg or 150 mg.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments will become apparent from a review of the ensuing detailed description.

### BRIEF DESCRIPTION OF THE DRAWINGS

FIG. 1 illustrates reductions in serum IgG concentrations following treatment of highly sensitized chronic kidney disease patients with REGN5459, as discussed in Example 2.

FIGS. 2A and 2B show the experimental setup to assess in vivo targeting of plasma cells by BCMA x CD3 bispecific antibody in BCMAhu/huCD3hu/hu mice (FIG. 2A) and the quantification of BMPCs (FIG. 2B).

FIGS. 3A and 3B show the levels of IgA+ (FIG. 3A) and IgM+ (FIG. 3B) BMPCs upon administration of REGN5459 in BCMAhu/huCD3hu/hu mice.

FIG. 4 shows the quantification of splenic antigen-experienced B cells 7 days after BCMA x CD3 bispecific antibody administration in BCMAhu/huCD3hu/hu mice.

FIGS. 5A, 5B and 5C show the quantification of Serum IgA (FIG. 5A), IgM (FIG. 5B) and IgG1 (FIG. 5C) levels after BCMA x CD3 bispecific antibody administration in BCMAhu/huCD3hu/hu mice.

FIGS. 6A, 6B and 6C depict the study design showing 15 weeks of continuous HDM exposure with continuous anti-IL-4Ra starting at week 12 and BCMAxCD3 dosed transiently at week 15 (FIG. 6A); quantification of IgE+BMPCs as percent of live cells (FIG. 6B); and quantification of total BMPCs (FIG. 6C).

FIGS. 7A and 7B show Cynomolgus monkey study design (FIG. 7A). Anti-IL-4Ra was dosed weekly starting on day 1, and a single dose of BCMAxCD3 or isotype control antibody was administered on day 22. Bone marrow aspirates were taken at day 15 and day 43. (FIG. 7B) Flow cytometry quantification of BMPCs from bone marrow aspirates sampled at the noted time points.

FIGS. 8A, 8B and 8C show the mean concentrations of serum IgG (FIG. 8B) and IgE (FIG. 8C) levels over time in multiple myeloma patients receiving weekly BCMAxCD3 bispecific antibody at the indicated dose levels (FIG. 8A).

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

The expression “CD3,” as used herein, refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma. Human CD3-epsilon comprises the amino acid sequence as set forth in SEQ ID NO: 23; human CD3-delta comprises the amino acid sequence as set forth in SEQ ID NO: 24; human CD3-zeta comprises the amino acid sequence as set forth in SEQ ID NO: 25; and CD3-gamma comprises the amino acid sequence as set forth in SEQ ID NO: 26. All references to proteins, polypeptides and protein fragments herein are intended to refer to the human version of the respective protein, polypeptide or protein fragment unless explicitly specified as being from a non-human species. Thus, the expression “CD3” means human CD3 unless specified as being from a non-human species, e.g., “mouse CD3,” “monkey CD3,” etc.

As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). The antibodies and antigen-binding fragments of the present invention may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression, “cell surface-expressed CD3” also includes a CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

The expression “BCMA,” as used herein, refers to B-cell maturation antigen. BCMA (also known as TNFRSF17 and CD269) is a cell surface protein expressed on plasma cells, and plays a central role in regulating B cell maturation and differentiation into immunoglobulin-producing plasma cells. The amino acid sequence of human BCMA is shown in SEQ ID NO: 22, and can also be found in GenBank accession number NP_001183.2.

As used herein, “an antibody that binds BCMA” or an “anti-BCMA antibody” includes antibodies and antigen-binding fragments thereof that specifically recognize BCMA.

A “plasma cell” is a differentiated B-lymphocyte capable of secreting antibodies, including alloantibodies.

“Human leukocyte antigens” (HLAs) are cell surface proteins that present peptides to T lymphocytes as part of the immune recognition process, which is fundamental to the adaptive immune response, and the genes that encode the HLAs are among the most polymorphic genes in the human genome. Sensitization, the presence of anti-HLA antibodies, occurs in ˜30% of patients in need of solid organ transplants. These alloantibodies develop after exposure to nonself HLA through pregnancy, blood transfusion, or a previous organ transplant.

An “alloantibody” is an antibody produced in response to exposure to incompatible blood group antigens, and is directed against a nonself HLA protein.

“Calculated panel reactive antibody” (cPRA) level is a value based on the HLA antigens that are listed as unaccepable for solid organ (e.g., kidney) transplant candidates. cPRA represents a method for determining a patient's risk of organ rejection prior to transplantation, and is an estimate of the percentage of donors with whom a particular recipient would be incompatible. A cPRA>80% is considered highly sensitized.

A “subject,” as used herein, refers an individual (e.g., a human) in need of a solid organ transplant (e.g., a kidney transplant) who is at risk of antibody-mediated rejection of the transplanted organ due to the presence of alloantibodies. The subject may be an individual with chronic kidney disease, or an individual with chronic kidney disease that requires hemodialysis.

“Chronic Kidney Disease” or CKD, as used herein, refers to a condition characterized by decreased kidney function, either because of kidney damage or a decreased glomerular filtration rate (GFR) of less than 60 mL/min/1.73 m² for at least 3 months. Different stages of CKD define a continuum, classified as: Stage 1 (kidney damage with normal GFR); Stage 2 (mild reduction in GFR to 60-89 mL/min/1.73 m²); Stage 3a (moderate reduction in GFR to 45-59 mL/min/1.73 m²); Stage 3b (moderate reduction in GFR to 30-44 mL/min/1.73 m²); Stage 4 (severe reduction in GFR to 15-29 mL/min/1.73 m²); and Stage 5 (kidney failure and a GFR of <15 mL/min/1.73 m² or dialysis).

A “control population,” as used herein, refers to a group of individuals for which a mean calculated or measured parameter can be used as a comparator. For example, a control population with a pre-transplant cPRA level at a specified value (e.g., 90%) refers to a group of individuals with a mean cPRA level at the specified value, and the control population's cPRA level can be used as a comparator to evaluate the impact of antibody therapy (e.g., to reduce alloantibody levels or reduce of risk of antibody-mediated graft rejection) as discussed herein.

The term “antigen-binding molecule” includes antibodies and antigen-binding fragments of antibodies, including, e.g., bispecific antibodies.

The term “antibody”, as used herein, means any antigen-binding molecule or molecular complex comprising at least one complementarity determining region (CDR) that specifically binds to or interacts with a particular antigen (e.g., BCMA or CD3). The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds. Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-BCMA antibody or anti-CD3 antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The antibodies of the present invention may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments of the invention, the anti-BCMA x anti-CD3 bispecific antibodies of the invention are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-BCMA x anti-CD3 antibodies discussed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-BCMA x anti-CD3 antibodies comprising variants of any of the HCVR, LCVR, and/or CDR amino acid sequences disclosed herein having one or more conservative substitutions. For example, the present invention includes anti-BCMA x anti-CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth herein, or the full-length heavy chain and light chain sequences noted herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear.

A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule. In some cases, the bispecific antibodies or antigen-binding fragments thereof are 90%, 95%, 96%, 97%, 98%, or 99% identical to the sequences discussed herein (e.g., the CDR, HCVR, LCVR, heavy chain, or light chain sequences).

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

As used herein, the term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, the antibody or antigen-binding protein of the invention binds to the predetermined antigen or cell surface molecule (receptor) having an affinity corresponding to a K_{D} value that is at least ten-fold lower than its affinity for binding to a non-specific antigen (e.g., BSA, casein). According to the present invention, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody of the invention.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec−1 or 1/s) refers to the dissociation rate constant of a particular antibody-antigen interaction, or the dissociation rate constant of an antibody or antibody-binding fragment. Said value is also referred to as the k_{off} value.

The term “kₐ” (M-1×sec-1 or 1/M) refers to the association rate constant of a particular antibody-antigen interaction, or the association rate constant of an antibody or antibody-binding fragment.

The term “K_{A}” (M-1 or 1/M) refers to the association equilibrium constant of a particular antibody-antigen interaction, or the association equilibrium constant of an antibody or antibody-binding fragment. The association equilibrium constant is obtained by dividing the kₐ by the k_{d}.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody of the invention that gives half-maximal binding to cells expressing CD3 or BCMA, as determined by e.g. a FACS binding assay. Thus, reduced or weaker binding is observed with an increased EC₅₀, or half maximal effective concentration value.

In one embodiment, decreased binding can be defined as an increased EC₅₀ antibody concentration which enables binding to the half-maximal amount of target cells.

In another embodiment, the EC₅₀ value represents the concentration of an antibody of the invention that elicits half-maximal depletion of target cells by T cell cytotoxic activity. Thus, increased cytotoxic activity (e.g. T cell-mediated plasma cell killing) is observed with a decreased EC₅₀, or half maximal effective concentration value.

### Methods for Desensitizing Patients to Anti-HLA Alloantibodies to Facilitate Successful Solid Organ Transplantation

The present disclosure provides methods of using anti-BCMA x anti-CD3 bispecific antibodies (e.g., REGN5459 or REGN5458) to safely deplete B cell maturation antigen (BCMA)-expressing cells (e.g., plasma cells) and decrease anti-HLA alloantibodies to facilitate solid organ transplantation (e.g., kidney transplantation). Plasma cell-targeting therapies (as described herein), by eliminating the source of anti-HLA alloantibodies, will have a longer-lasting HLA desensitization effect, facilitating transplantation and reducing the risk of post-transplant antibody-mediated rejection (AMR). Anti-BCMA x CD3 antibodies (e.g., REGN5459 and REGN5458) target the surface protein BCMA that is selectively expressed on plasma cells. The bispecific antibodies deplete plasma cells and newly activated BCMA-expressing B cells, including those that produce anti-HLA antibodies, without impairing regulatory T cell responses that promote tolerance after a transplant. Desensitization of patients to anti-HLA alloantibodies may be measured, for example, by reducing alloantibody levels in a subject, by reducing a calculated panel reactive antibody (cPRA) level in a subject, or by comparing a subject's risk of rejection to a control population at the time of transplant or within specified periods following transplantation.

Desensitzation can be accomplished, e.g., via a method of reducing an alloantibody level in a subject in need of a solid organ transplant, in which the method comprises administering to the subject a bispecific antibody or antigen-binding fragment thereof comprising a first antigen-binding domain that specifically binds a human B-cell maturation antigen (BCMA) on a cell (e.g., a plasma cell), and a second antigen-binding domain that specifically binds human CD3 on a T cell. Exemplary bispecific antibodies and antigen-binding fragments thereof that can be used in connection with such a method are discussed in greater detail herein.

In some embodiments, reducing the alloantibody level in the subject comprises reducing the alloantibody level below a baseline alloantibody level measured prior to administration of the bispecific antibody or antigen-binding fragment thereof. In some cases, the method further comprises measuring a baseline alloantibody level in the subject prior to administration of the bispecific antibody or antigen-binding fragment thereof. In some cases, the alloantibody level and/or the baseline alloantibody level is measured by a single antigen bead assay, and the alloantibody level and/or the baseline alloantibody level corresponds to a peak immunodominant anti-HLA antibody mean fluorescence intensity. In some cases, the reduction in the alloantibody level corresponds to a reduction in a peak immunodominant anti-HLA antibody mean fluorescence intensity (MFI) by ≥50% relative to the baseline alloantibody level. In some cases, the reduction in the alloantibody level corresponds to a reduction in a peak immunodominant anti-HLA antibody MFI by, or by at least, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the alloantibody level is measured by a single antigen bead assay, and a reduction in the alloantibody level corresponds to a reduction in a peak immunodominant anti-HLA antibody mean fluorescence intensity to <5000. In some cases, the alloantibody level is measured by a single antigen bead assay, and a reduction in the alloantibody level corresponds to a reduction in a peak immunodominant anti-HLA antibody mean fluorescence intensity to <10000 m <9500, <9000, <8500, <8000, <7500, <7000, <6500, <6000, <5500, <5000, <4900, <4800, <4700, <4600, <4500, <4400, <4300, <4200, <4100, <4000, <3900, <3800, <3700, <3600, <3500, <3400, <3300, <3200, <3100, <3000, <2900, <2800, <2700, <2600, <2500, <2400, <2300, <2200, <2100, <2000, <1900, <1800, <1700, <1600, <1500, <1400, <1300, <1200, <1100, <1000, <900, <800, <700, <600, <500, <490, <480, <470, <460, or <450.

Desensitzation can be accomplished, e.g., via a method of reducing a calculated panel reactive antibody (cPRA) level of a subject in need of a solid organ transplant, in which the method comprises administering to the subject a bispecific antibody or antigen-binding fragment thereof comprising a first antigen-binding domain that specifically binds a human B-cell maturation antigen (BCMA) on a cell (e.g., a plasma cell), and a second antigen-binding domain that specifically binds human CD3 on a T cell. Exemplary bispecific antibodies and antigen-binding fragments thereof that can be used in connection with such a method are discussed in greater detail herein.

In some embodiments, reducing the cPRA level of the subject comprises reducing the cPRA level of the subject below a baseline cPRA level determined prior to administration of the bispecific antibody or antigen-binding fragment thereof. In some cases, the cPRA level of the subject is reduced to: <99%; <98%; <97%; <96%; <95%; <94%; <93%; <92%; <91%; <90%; <89%; <88%; <87%; <86%; <85%; <84%; <83%; <82%; <81%; or <80%. In some cases, the cPRA level of the subject is reduced to, or reduced to less than: 99.5%, 99%, 98.5%, 98%, 97.5%, 97%, 96.5%, 96%, 95.5%, 95%, 94.5%, 94%, 93.5%, 93%, 92.5%, 92%, 91.5%, 91%, 90.5%, 90%, 89.5%, 89%, 88.5%, 88%, 87.5%, 87%, 86.5%, 86%, 85.5%, 85%, 84.5%, 84%, 83.5%, 83%, 82.5%, 82%, 81.5%, 81%, 80.5%, 80%, 79.5%, 79%, 78.5%, 78%, 77.5%, 77%, 76.5%, 76%, 75.5%, 75%, 74.5%, 74%, 73.5%, 73%, 72.5%, 72%, 71.5%, 71%, 70.5%, 70%, 69.5%, 69%, 68.5%, 68%, 67.5%, 67%, 66.5%, 66%, 65.5%, 65%, 64.5%, 64%, 63.5%, 63%, 62.5%, 62%, 61.5%, 61%, 60.5%, 60%, 59.5%, 59%, 58.5%, 58%, 57.5%, 57%, 56.5%, 56%, 55.5%, 55%, 54.5%, 54%, 53.5%, 53%, 52.5%, 52%, 51.5%, 51%, 50.5%, 50%, 45%, 40%, 35%, 30%, 25%, 20%, 15%, or 10%.

The present disclosure also provides a method of reducing sensitization of a subject to anti-HLA antibodies prior to solid organ transplant, in which the method comprises administering to the subject a bispecific antibody or antigen-binding fragment thereof comprising a first antigen-binding domain that specifically binds a human B-cell maturation antigen (BCMA) on a cell (e.g., a plasma cell), and a second antigen-binding domain that specifically binds human CD3 on a T cell, wherein the subject's risk of rejection at the time of organ transplant is no greater than that of a control population with a pre-transplant calculated panel reactive antibody (cPRA) level of 90%. Exemplary bispecific antibodies and antigen-binding fragments thereof that can be used in connection with such a method are discussed in greater detail herein.

In some embodiments, the subject's risk of rejection at the time of organ transplant is no greater than that of a control population with a pre-transplant cPRA level of 80%. In some embodiments, the subject's risk of rejection at the time of organ transplant is no greater than that of a control population with a pre-transplant cPRA level of from 50% to <80%. In some embodiments, the subject's risk of rejection at the time of organ transplant is no greater than that of a control population with a pre-transplant cPRA level of 95%, 94.5%, 94%, 93.5%, 93%, 92.5%, 92%, 91.5%, 91%, 90.5%, 90%, 89.5%, 89%, 88.5%, 88%, 87.5%, 87%, 86.5%, 86%, 85.5%, 85%, 84.5%, 84%, 83.5%, 83%, 82.5%, 82%, 81.5%, 81%, 80.5%, 80%, 79.5%, 79%, 78.5%, 78%, 77.5%, 77%, 76.5%, 76%, 75.5%, 75%, 74.5%, 74%, 73.5%, 73%, 72.5%, 72%, 71.5%, 71%, 70.5%, 70%, 69.5%, 69%, 68.5%, 68%, 67.5%, 67%, 66.5%, 66%, 65.5%, 65%, 64.5%, 64%, 63.5%, 63%, 62.5%, 62%, 61.5%, 61%, 60.5%, 60%, 59.5%, 59%, 58.5%, 58%, 57.5%, 57%, 56.5%, 56%, 55.5%, 55%, 54.5%, 54%, 53.5%, 53%, 52.5%, 52%, 51.5%, 51%, 50.5%, 50%, 45%, 40%, 35%, 30%, 25%, 20%, 15%, or 10%.

The present disclosure also provides a method of reducing risk of allograft rejection in a subject following a solid organ transplant, the method comprising administering to the subject a bispecific antibody or antigen-binding fragment thereof comprising a first antigen-binding domain that specifically binds a human B-cell maturation antigen (BCMA) on a cell (e.g., a plasma cell), and a second antigen-binding domain that specifically binds human CD3 on a T cell, wherein the subject's risk of rejection during a post-transplant interval is no greater than that of a control population with a pre-transplant calculated panel reactive antibody (cPRA) level of 90%. Exemplary bispecific antibodies and antigen-binding fragments thereof that can be used in connection with such a method are discussed in greater detail herein.

In some embodiments, the subject's risk of rejection during the post-transplant interval is no greater than that of a control population with a pre-transplant cPRA level of 80%. In some embodiments, the subject's risk of rejection during the post-transplant interval is no greater than that of a control population with a pre-transplant cPRA level of from 50% to <80%. In some embodiments, the subject's risk of rejection during the post-transplant interval is no greater than that of a control population with a pre-transplant cPRA level of 95%, 94.5%, 94%, 93.5%, 93%, 92.5%, 92%, 91.5%, 91%, 90.5%, 90%, 89.5%, 89%, 88.5%, 88%, 87.5%, 87%, 86.5%, 86%, 85.5%, 85%, 84.5%, 84%, 83.5%, 83%, 82.5%, 82%, 81.5%, 81%, 80.5%, 80%, 79.5%, 79%, 78.5%, 78%, 77.5%, 77%, 76.5%, 76%, 75.5%, 75%, 74.5%, 74%, 73.5%, 73%, 72.5%, 72%, 71.5%, 71%, 70.5%, 70%, 69.5%, 69%, 68.5%, 68%, 67.5%, 67%, 66.5%, 66%, 65.5%, 65%, 64.5%, 64%, 63.5%, 63%, 62.5%, 62%, 61.5%, 61%, 60.5%, 60%, 59.5%, 59%, 58.5%, 58%, 57.5%, 57%, 56.5%, 56%, 55.5%, 55%, 54.5%, 54%, 53.5%, 53%, 52.5%, 52%, 51.5%, 51%, 50.5%, 50%, 45%, 40%, 35%, 30%, 25%, 20%, 15%, or 10%. In some cases, the post-transplant interval is a 3-month interval beginning one day after organ transplant. In some cases, the post-transplant interval is a 6-month interval beginning one day after organ transplant. In some cases, the post-transplant interval is a 12-month interval beginning one day after organ transplant. In various embodiments, the post-transplant interval is a period beginning one day after organ transplant and ending after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months.

In some embodiments, graft function is maintained in the subject during the post-transplant interval at a function level equal to or greater than that of the control population.

In any of the various embodiments of the methods discussed above or herein, the subject may be a human.

In any of the various embodiments of the methods discussed above or herein, the solid organ may be selected from the group consisting of a kidney, a lung, a pancreas, a heart, a colon or portion thereof, a liver or portion thereof, or skin. In some cases, the solid organ is a kidney.

The methods discussed herein address a high unmet need to facilitate solid organ transplantation (e.g., kidney transplantation) for patients highly sensitized to HLA. Using kidney transplantation as an example, due to the difficulty of finding a compatible organ donor, less than 10% of highly sensitized patients in need of a kidney will receive a kidney transplant. Patients who are highly sensitized to HLA are frequently defined as having a cPRA of ≥80%. The cPRA score represents the probability of encountering an incompatible donor for organ transplant candidates and is used as a measurement of sensitization level. While the kidney allocation system has improved transplantation rates for highly sensitized individuals overall, 30% to 50% of patients with a cPRA score ≥90% remain on the kidney transplant waitlist for over 5 years, highlighting the unmet need for this subset of sensitized patients. Of the approximately 11,000 chronic kidney disease patients with a cPRA≥90% who are on the kidney transplant waitlist in the US, the majority (approximately 7,000 patients) have cPRA scores >98%. These patients have a particularly high unmet need as evidenced by their more frequent removal from the waitlist, and death, due to medical comorbidities. Furthermore, for the approximately 2,000 patients on the kidney transplant waitlist with a cPRA≥99.9%, the average rate of transplantation is markedly lower than for other HLA-sensitized patients, despite the implementation of the kidney allocation system in 2014. Patients highly sensitized to HLA spend years dependent on hemodialysis, which is associated with a poor quality of life, high rates of illness, and a mortality rate of up to 20% to 25% while on the kidney transplant waitlist; therefore, a treatment that increases the probability of kidney transplantation is advantageous. In some cases, the methods discussed herein may be applied to a subject seeking a solid organ transplant that have been on a waitlist (e.g., a kidney transplant waitlist) for at least 1 year, at least 2 years, at least 3 years, at least 4 years, or at least 5 years, or longer. Although desensitization according to the methods discussed herein will not necessarily lead to an organ transplant because of limited availability, a decrease in the number of unacceptable HLA matches will improve each patient's likelihood of a transplant offer. Furthermore, patients who are offered an organ during the study, and have an acceptable crossmatch, may receive an organ (e.g., kidney) transplant.

Plasma cells are a promising target for the treatment of antibody-mediated autoimmune and alloimmune disease, including desensitization of individuals who are highly sensitized to HLA, preventing them from safely obtaining a transplant. Due to the elimination of the source of anti-HLA alloantibodies, plasma cell-targeting therapies may have a longer-lasting HLA desensitization effect than plasmapheresis and other antibody-reducing treatments. Bispecific anti-BCMA x anti-CD3 antibodies, including REGN5459 and REGN5458, can be used to bind to BCMA on plasma cells and to CD3 on T cells. While both REGN5459 and REGN5458 contain the same BCMA-binding arm, the difference between these two molecules is their respective affinities for CD3, where REGN5459 binds CD3 on T cells with a lower affinity than that of REGN5458. In vitro, each of these bispecific antibodies triggers activation of Jurkat T lymphocytes only in the presence of human BCMA-expressing cells. REGN5459 and REGN5458 induce T cell activation and cytotoxicity directed toward tumor cells with a broad range of BCMA protein expression levels; the cell surface expression levels of BCMA on target cells does not affect the potency of either REGN5459 or REGN5458. REGN5459 and REGN5458 can mediate the killing of multiple cell lines and primary cells with a range of BCMA cell surface expression, including normal plasma cells in the bone marrow and secondary lymphoid organs, as demonstrated in healthy Cynomolgus macaques. Due to its lower affinity for CD3, REGN5459 is hypothesized to have a lower likelihood of eliciting CRS, with similar efficacy in plasma cell depletion.

Clinically relevant metrics used for sensitized patients in need of a solid organ (e.g., kidney) transplant include the breadth of reactivity of donor-specific antibodies (DSAs), in particular, of anti-HLA alloantibodies measured in a Luminex-based single antigen bead (SAB) assay. The SAB assay measures reactivity of the patient's serum or plasma to a panel of HLA-coated beads, which is expressed as mean fluorescence intensity (MFI). By using center-specific cutoff values and algorithms, HLA proteins are identified on a per-patient basis for which the individual has an unacceptable threshold level of preformed alloantibodies. The patient's own HLA genotype is considered to distinguish bona fide alloreactivity from the background fluorescence signal. Based on the number of unacceptable HLA matches and estimated HLA allele frequency within the relevant population, the cPRA level (range: 0% to 100%), which represents the probability of encountering an incompatible donor for organ transplant, can be computed and used as a measurement of sensitization level.

Because anti-HLA alloantibodies are well characterized prognostic biomarkers and can predict clinical outcomes for solid organ (e.g., kidney) transplants, anti-HLA monitoring is part of the consensus guidelines established by the Antibody Consensus Group of the Transplantation Society. The risks of AMR and graft loss significantly increase by the presence of anti-HLA alloantibodies against donor-specific HLA above a certain threshold represented as MFI. A peak anti-HLA MFI>3,000 has been associated with a significant increase in AMR, and decreased 1-, 3-, and 5-year graft survival, while patients with anti-HLA MFIs of 465 to approximately 3,000 have shown similar rates of AMR and graft survival to a control group with almost no anti-HLA (highest MFI≤465). Other studies have also found significantly increased risk of post-transplantation AMR in patients with pretransplant peak anti-HLA MFI of >3,000 or >8,000. Thus, there is a clear correlation between high-peak anti-HLA MFI, AMR, and decreased graft survival; anti-HLA MFI>3,000 to 5,000 is generally considered high-risk, and MFI>10,000 is almost universally considered unacceptable for a transplant from a patient with the HLA type (without desensitization). In addition, there are other less-standardized DSA detection techniques, including measuring the titer of anti-HLA alloantibodies by serially diluting serum samples for SAB assays; specific measurement of C1q-binding anti-HLA alloantibodies using a similar Luminex platform; and cell-based physical crossmatch assays against T cells (representing class I HLA), B cells (representing class II HLA), and endothelial precursor cells (used to test reactivity against non-HLA antigens). Cell-based crossmatch assays can be flow cytometry-based assay and cytotoxicity-based assay.

Although plasma cells are the target cells for the anti-BCMA x anti-CD3 antibodies (e.g., REGN5459 and REGN5458), the rarity of these cells in circulation may limit reliable quantification. Thus, concentrations of Ig subclasses (IgA, IgM, IgE, IgG, IgG1, IgG2 and IgG3) can be monitored, as well as pathogen-specific antibody titers, as potential surrogates for antibody-producing plasma cell levels. Desensitzied patients treated according the methods discussed herein, and who receive a solid organ (e.g., kidney) transplant may be followed for a period of time (e.g., 12 months) post-transplant with no further bispecific anti-BCMA x CD3 antibody administered to assess post-transplant outcomes. Graft function after transplantation (e.g., kidney transplantation) will be the primary measure of success of the transplant, and the incidence of graft loss and delayed graft function may be determined. Incidence of allograft rejection, histologic form of rejection (cell-mediated vs. antibody-mediated vs. mixed) and Banff classification of kidney allograft pathology may also be determined from kidney biopsies obtained as standard of care or for-cause when rejection is suspected (e.g., in the setting of a rising serum creatinine, worsening hypertension, increased proteinuria). Anti-HLA MFI and cPRA may also be determined, as these biomarker surrogates correlate highly with relevant clinical outcomes, including post-transplant AMR and graft survival. The incidence of CMV, EBV, and BKV reactivation and infection will also be assessed since these viruses are common post-transplant, and the impact of plasma cell-depleting therapy on their incidence is unknown.

In the context of kidney transplantation, the incidence of delayed graft function, defined as the need for dialysis within 7 days of transplantation, may be assessed as it is associated with higher rates of acute rejection and is one of the strongest risk factors for chronic allograft nephropathy, translating to a 40% decrease in long-term graft survival. The incidences and classification of biopsy-proven TCMR and AMR may be monitored, as both types of rejection alter graft histology and negatively impact graft survival. Moreover, since 25% to 45% of highly-sensitized patients experience AMR post-transplant and 80% of these AMR episodes occur in the first month post-transplant, evaluation of the preliminary rates of AMR in sensitized transplant recipients previously treated with REGN5459 or REGN5458 may be undertaken. Finally, estimated glomerular filtration rate (eGFR) over time may be monitored as a critical functional metric of graft function post-transplant.

### Combination Therapies

The bispecific anti-BCMA x anti-CD3 antibodies and antigen-binding fragments thereof discussed herein may be combined with other therapeutics to desensitize patients to anti-HLA alloantibodies. Such combinations may include, in addition to the bispecific antibody, the use of IVIG and/or plasmapheresis. Protocols that combine plasmapheresis and low-dose IVIG (100 mg/kg) have demonstrated a transient reduction in anti-HLA levels and, in some cases, facilitated transplantation after repeated administration, depending on the response of anti-HLA alloantibodies. Other combinations may include, in addition to the bispecific antibody, rituximab, antibody blockers of interleukin (IL)-6/IL-6 receptor (e.g., clazakizumab/tocilizumab), proteasome inhibitors (e.g., carfilzomib, bortezomib), other plasma cell-targeting therapies (e.g., daratumumab), and imlifidase/IgG-degrading enzyme from Streptococcus pyogenes (IdeS).

The present invention provides methods which comprise administering a pharmaceutical composition comprising any of the exemplary bispecific antigen-binding molecules described herein in combination with one or more additional therapeutic agents. The additional therapeutically active component(s) may be administered just prior to, concurrent with, or shortly after the administration of a bispecific antigen-binding molecule of the present invention; (for purposes of the present disclosure, such administration regimens are considered the administration of a bispecific antigen-binding molecule “in combination with” an additional therapeutically active component).

The present invention includes pharmaceutical compositions in which a bispecific antigen-binding molecule of the present invention is co-formulated with one or more of the additional therapeutically active component(s) discussed herein.

### Bispecific Antibodies and Antigen-Binding Fragments Thereof

The present invention includes bispecific antigen-binding molecules that specifically bind CD3 and BCMA. Such molecules may be referred to herein as, e.g., “anti-BCMA x anti-CD3” or “anti-CD3/anti-BCMA,” or “anti-CD3xBCMA” or “CD3xBCMA” bispecific molecules, or other similar terminology (e.g., anti-BCMA/anti-CD3).

In certain embodiments, the CD3-binding arm binds to human CD3 and induces human T cell activation. In certain embodiments, the CD3-binding arm binds weakly to human CD3 and induces human T cell activation. In other embodiments, the CD3-binding arm binds weakly to human CD3 and induces BCMA-expressing cell killing. In other embodiments, the CD3-binding arm binds or associates weakly with human and cynomolgus (monkey) CD3, yet the binding interaction is not detectable by in vitro assays known in the art.

The term “BCMA,” as used herein, refers to the human BCMA protein unless specified as being from a non-human species (e.g., “mouse BCMA,” “monkey BCMA,” etc.). The human BCMA protein has the amino acid sequence shown in SEQ ID NO: 22.

The aforementioned bispecific antigen-binding molecules that specifically bind CD3 and BCMA may comprise an anti-CD3 antigen-binding molecule which binds to CD3 with a weak binding affinity such as exhibiting a K_{D} of greater than about 40 nM, as measured by an in vitro affinity binding assay.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present invention, the first antigen-binding domain specifically binds a first antigen (e.g., BCMA), and the second antigen-binding domain specifically binds a second, distinct antigen (e.g., CD3).

In certain exemplary embodiments of the present invention, the bispecific antigen-binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “D1” and the CDRs of the second antigen-binding domain may be designated with the prefix “D2”. Thus, the CDRs of the first antigen-binding domain may be referred to herein as D1-HCDR1, D1-HCDR2, and D1-HCDR3; and the CDRs of the second antigen-binding domain may be referred to herein as D2-HCDR1, D2-HCDR2, and D2-HCDR3.

In certain exemplary embodiments, the isolated bispecific antigen binding molecule comprises a first antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 1; and (b) three light chain complementarity determining regions (LCDR1, LCDR2 and LCDR3) contained within a light chain variable region (LCVR) comprising the amino acid sequence of SEQ ID NO:5. In some cases, the isolated bispecific antigen binding molecule comprises a HCDR1 comprising the amino acid sequence of SEQ ID NO:2, a HCDR2 comprising the amino acid sequence of SEQ ID NO:3, and a HCDR3 comprising the amino acid sequence of SEQ ID NO:4. In some cases, the isolated bispecific antigen-binding molecule comprises a LCDR1 comprising the amino acid sequence of SEQ ID NO:6, a LCDR2 comprising the amino acid sequence of SEQ ID NO:7, and a LCDR3 comprising the amino acid sequence of SEQ ID NO:8. In some cases, the first antigen-binding domain comprises a HCVR comprising the amino acid sequence of SEQ ID NO: 1, and a LCVR comprising the amino acid sequence of SEQ ID NO: 5.

In certain exemplary embodiments, the isolated bispecific antigen-binding molecule comprises a second antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 9 or SEQ ID NO: 13; and (b) three light chain complementarity determining regions (LCDR1, LCDR2 and LCDR3) contained within a light chain variable region (LCVR) comprising the amino acid sequence of SEQ ID NO:5. In some cases, the second antigen-binding domain comprises: (a) a HCDR1 comprising the amino acid sequence of SEQ ID NO: 10 or SEQ ID NO: 14; (b) a HCDR2 comprising the amino acid sequence of SEQ ID NO: 11 or SEQ ID NO: 15; and (c) a HCDR3 comprising the amino acid sequence of SEQ ID NO: 12 or SEQ ID NO: 16. In some cases, the second antigen-binding domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8. In some cases, the second antigen-binding domain comprises: (a) HCDR1, HCDR2, HCDR3 domains, respectively, comprising the amino acid sequences of SEQ ID NOs: 10, 11, 12; and LCDR1, LCDR2, LCDR3 domains, respectively, comprising the amino acid sequences of SEQ ID NOs: 6, 7, 8; or (b) HCDR1, HCDR2, HCDR3 domains, respectively, comprising the amino acid sequences of SEQ ID NOs: 14, 15, 16; and LCDR1, LCDR2, LCDR3 domains, respectively, comprising the amino acid sequences of SEQ ID NOs: 6, 7, 8. In some cases, the second antigen-binding domain comprises: (a) a HCVR comprising the amino acid sequence of SEQ ID NO: 9, and a LCVR comprising the amino acid sequence of SEQ ID NO: 5; or (b) a HCVR comprising the amino acid sequence of SEQ ID NO: 13, and a LCVR comprising the amino acid sequence of SEQ ID NO: 5.

In certain exemplary embodiments, the isolated bispecific antigen-binding molecule comprises: (a) a first antigen-binding domain that comprises HCDR1, HCDR2, HCDR3 domains, respectively, comprising the amino acid sequences of SEQ ID NOs: 2, 3, 4, and LCDR1, LCDR2, LCDR3 domains, respectively, comprising the amino acid sequences of SEQ ID NOs: 6, 7, 8; and (b) a second antigen binding domain that comprises HCDR1, HCDR2, HCDR3 domains, respectively, comprising the amino acid sequences of SEQ ID NOs: 10, 11, 12, and LCDR1, LCDR2, LCDR3 domains, respectively, comprising the amino acid sequences of SEQ ID NOs: 6, 7, 8. In some cases, the isolated bispecific antigen-binding molecule comprises: (a) a first antigen binding domain that comprises a HCVR comprising the amino acid sequence of SEQ ID NO: 1, and a LCVR comprising the amino acid sequence of SEQ ID NO: 5; and (b) a second antigen binding domain that comprises a HCVR comprising the amino acid sequence of SEQ ID NO: 9, and a LCVR comprising the amino acid sequence of SEQ ID NO: 5.

In certain exemplary embodiments, the isolated bispecific antigen-binding molecule comprises: (a) a first antigen-binding domain that comprises HCDR1, HCDR2, HCDR3 domains, respectively, comprising the amino acid sequences of SEQ ID NOs: 2, 3, 4, and LCDR1, LCDR2, LCDR3 domains, respectively, comprising the amino acid sequences of SEQ ID NOs: 6, 7, 8; and (b) a second antigen binding domain that comprises HCDR1, HCDR2, HCDR3 domains, respectively, comprising the amino acid sequences of SEQ ID NOs: 14, 15, 16, and LCDR1, LCDR2, LCDR3 domains, respectively, comprising the amino acid sequences of SEQ ID NOs: 6, 7, 8. In some cases, the isolated bispecific antigen-binding molecule comprises: (a) a first antigen binding domain that comprises a HCVR comprising the amino acid sequence of SEQ ID NO: 1, and a LCVR comprising the amino acid sequence of SEQ ID NO: 5; and (b) a second antigen binding domain that comprises a HCVR comprising the amino acid sequence of SEQ ID NO: 13, and a LCVR comprising the amino acid sequence of SEQ ID NO: 5.

An exemplary bispecific antigen-binding molecule comprises: (a) a first antigen binding domain that comprises a HCVR comprising the amino acid sequence of SEQ ID NO: 1, and a LCVR comprising the amino acid sequence of SEQ ID NO: 5; and (b) a second antigen binding domain that comprises a HCVR comprising the amino acid sequence of SEQ ID NO: 9, and a LCVR comprising the amino acid sequence of SEQ ID NO: 5 is the bispecific antibody. This antibody is also referred to as REGN5458.

An exemplary bispecific antigen-binding molecule comprises: (a) a first antigen binding domain that comprises a HCVR comprising the amino acid sequence of SEQ ID NO: 1, and a LCVR comprising the amino acid sequence of SEQ ID NO: 5; and (b) a second antigen binding domain that comprises a HCVR comprising the amino acid sequence of SEQ ID NO: 13, and a LCVR comprising the amino acid sequence of SEQ ID NO: 5 is the bispecific antibody. This antibody is also referred to as REGN5459.

In certain exemplary embodiments, the isolated bispecific antigen binding molecule competes for binding to BCMA, or binds to the same epitope on BCMA as a reference antibody, wherein the reference antibody comprises a first antigen-binding domain comprising an HCVR/LCVR pair comprising the amino acid sequences of SEQ ID NOs: 1/5 and a second antigen-binding domain comprising an HCVR/LCVR pair comprising the amino acid sequences of either SEQ ID NOs: 9/5 or SEQ ID NOs: 13/5.

In certain exemplary embodiments, the isolated bispecific antigen binding molecule competes for binding to human CD3, or binds to the same epitope on human CD3 as a reference antibody, wherein the reference antibody comprises a first antigen-binding domain comprising an HCVR/LCVR pair comprising the amino acid sequences of SEQ ID NOs: 1/5 and a second antigen-binding domain comprising an HCVR/LCVR pair comprising the amino acid sequences of either SEQ ID NOs: 9/5 or SEQ ID NOs: 13/5.

The bispecific antigen-binding molecules discussed above or herein may be bispecific antibodies. In some cases, the bispecific antibody comprises a human IgG heavy chain constant region. In some cases, the human IgG heavy chain constant region is of isotype IgG1, IgG2, IgG3 or IgG4. In some cases, the human IgG heavy chain constant region is isotype IgG1. In some cases, the human IgG heavy chain constant region is isotype IgG4. In various embodiments, the bispecific antibody comprises a chimeric hinge that reduces Fcγ receptor binding relative to a wild-type hinge of the same isotype.

The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules of the present invention will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antigen-binding molecules of the present invention, the multimerizing domains, e.g., Fc domains, may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

The present invention also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. In some embodiments, one heavy or the other, but not both, comprises a C_{H}3 domain with both a H435R and a Y436F modification. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fc domains that can be included in any of the antigen-binding molecules of the present invention are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function. In various embodiments, the bispecific antigen-binding molecules may comprise a heavy chain constant region including a hinge domain, in which positions 233-236 within the hinge domain may be G, G, G and unoccupied; G, G, unoccupied, and unoccupied; G, unoccupied, unoccupied, and unoccupied; or all unoccupied, with positions numbered by EU numbering.

### Sequence Variants

The antibodies and bispecific antigen-binding molecules of the present invention may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germline sequences available from, for example, public antibody sequence databases. The antigen-binding molecules of the present invention may comprise antigen-binding domains which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, improved or enhanced antagonistic or agonistic biological properties (as the case may be), reduced immunogenicity, etc. Bispecific antigen-binding molecules comprising one or more antigen-binding domains obtained in this general manner are encompassed within the present invention.

pH-Dependent Binding

The present invention includes anti-BCMA x anti-CD3 bispecific antigen-binding molecules, with pH-dependent binding characteristics. For example, the antibodies of the present invention may exhibit reduced binding to BCMA at acidic pH as compared to neutral pH. Alternatively, antibodies of the invention may exhibit enhanced binding to BCMA at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to BCMA at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Antibodies Comprising Fc Variants

According to certain embodiments of the present invention, anti-BCMA x anti-CD3 bispecific antigen-binding molecules are provided comprising an Fc domain comprising one or more mutations which enhance or diminish antibody binding to the FcRn receptor, e.g., at acidic pH as compared to neutral pH. For example, the present invention includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). All positions are noted in EU numbering.

For example, the present invention includes anti-BCMA x anti-CD3 bispecific antigen-binding molecules comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

### Bioequivalents

The methods of present invention encompass antigen-binding molecules having amino acid sequences that vary from those of the exemplary molecules disclosed herein but that retain the ability to bind CD3 and/or BCMA. Such variant molecules may comprise one or more additions, deletions, or substitutions of amino acids when compared to parent sequence, but exhibit biological activity that is essentially equivalent to that of the described bispecific antigen-binding molecules.

The present invention includes methods comprising administration of antigen-binding molecules that are bioequivalent to any of the exemplary antigen-binding molecules set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Therapeutic Formulation and Administration

The present invention provides pharmaceutical compositions comprising the antigen-binding molecules of the present invention. The pharmaceutical compositions of the invention are formulated with suitable carriers, excipients, diluents, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like. The preferred dose is typically calculated according to body weight or body surface area. When a bispecific antigen-binding molecule of the present invention is used for therapeutic purposes in an adult patient, it may be advantageous to intravenously administer the bispecific antigen-binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ | ∥ and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.05 mg to about 500 mg per dosage form in a unit dose; especially in the form of injection, it is preferred that the aforesaid antibody is contained in about 0.05 mg to about 150 mg.

### Administration Regimens

According to certain embodiments of the present invention, multiple doses of an antigen-binding molecule (e.g., a bispecific anti-BCMA x anti-CD3 antibody) may be administered to a subject over a defined time course. The methods according to this aspect of the invention comprise sequentially administering to a subject multiple doses of an antigen-binding molecule of the invention. As used herein, “sequentially administering” means that each dose of an antigen-binding molecule is administered to the subject at a different point in time, e.g., on different days separated by a predetermined interval (e.g., hours, days, weeks or months). The present invention includes methods which comprise sequentially administering to the patient a single initial dose of an antigen-binding molecule, followed by one or more secondary doses of the antigen-binding molecule, and optionally followed by one or more tertiary doses of the antigen-binding molecule.

The terms “initial dose,” “secondary doses,” and “tertiary doses,” refer to the temporal sequence of administration of the antigen-binding molecule of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antigen-binding molecule, but generally may differ from one another in terms of frequency of administration. In certain embodiments, however, the amount of an antigen-binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In any of the embodiments, the initial dose (e.g., first weekly dose) may be split into two doses administered on separate days (e.g., consecutive days) no more than three days apart. In any of the embodiments, the first nominal dose (i.e., the secondary dose) may be split into two doses administered on separate days (e.g., consecutive days) no more than three days apart. For example, if the initial dose or the secondary dose is 1 mg, the dose may be split into two 0.5 mg doses administered on, e.g., consecutive days, or on separate days no more than three days apart. In various embodiments, the dose (e.g., the dose administered weekly, as a single dose or as two split fractions of the dose) is, or is at least, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.5 mg, 2 mg, 2.5 mg, 3 mg, 3.5 mg, 4 mg, 4.5 mg, 5, mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 510 mg, 520 mg, 530 mg, 540 mg, 550 mg, 560 mg, 570 mg, 580 mg, 590 mr, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, 10 g, or more. Any of these amounts may be used to define a range for the initial, secondary or tertiary doses discussed herein and are encompassed within the scope of this disclosure. In some embodiments, all doses are given as single doses (e.g., single infusions), including the doses administered in weeks one and two of a dosing regimen. For example, an initial dose of from 0.05 mg to 5 mg may be administered as a single dose in week one, a secondary dose of from 0.15 mg to 25 mg mg may be administered as a single dose in week two, and a tertiary dose of from 0.5 mg to 150 mg may be administered as a single dose in week three. Additional doses may be administered (e.g., at the same dose as the tertiary dose) weekly thereafter for a period of time. In another example, an initial dose of from 0.05 mg to 5 mg may be administered as a single dose in week one, a secondary dose of from 0.15 mg to 25 mg may be administered as a single dose in week two, and a tertiary dose of from 0.5 mg to 150 mg may be administered as a single dose in week three. In another example, an initial dose of from 0.05 mg to 1 mg may be administered as a single dose in week one, a secondary dose of from 0.15 mg to 5 mg may be administered as a single dose in week two, and a tertiary dose of from 0.5 mg to 40 mg may be administered as a single dose in week three. In some cases, the dosing schedule may thereafter (e.g., following weeks 1-3) include administration every week, every two weeks, every three weeks, once per month, or the like.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies that Bind BCMA and CD3

The present invention employs bispecific antigen-binding molecules (e.g., bispecific antibodies) that bind CD3 and BCMA; such bispecific antigen-binding molecules are also referred to herein as “anti-BCMA x anti-CD3 or anti-CD3xBCMA or anti-BCMA x anti-CD3 bispecific molecules.” The anti-BCMA portion of the anti-BCMA x anti-CD3 bispecific molecule is useful for targeting plasma cells that express BCMA (also known as CD269), and the anti-CD3 portion of the bispecific molecule is useful for activating T-cells. The simultaneous binding of BCMA on a plasma cell and CD3 on a T-cell facilitates directed killing (cell lysis) of the targeted plasma cells by the activated T-cell.

Bispecific antibodies comprising an anti-BCMA-specific binding domain and an anti-CD3-specific binding domain were constructed using standard methodologies, wherein the anti-BCMA antigen binding domain and the anti-CD3 antigen binding domain each comprise different, distinct HCVRs paired with a common LCVR. In exemplified bispecific antibodies, the molecules were constructed utilizing a heavy chain from an anti-CD3 antibody, a heavy chain from an anti-BCMA antibody and a common light chain from the anti-CD3 antibody (e.g., SEQ ID NO: 5). In other instances, the bispecific antibodies may be constructed utilizing a heavy chain from an anti-CD3 antibody, a heavy chain from an anti-BCMA antibody and an antibody light chain known to be promiscuous or pair effectively with a variety of heavy chain arms. Additional details regarding the anti-CD3 portions of the bispecific antibodies can be found in WO 2017/053856, which is herein incorporated by reference

TABLE 1
Summary of Component Parts of Anti-BCMA ×
Anti-CD3 Bispecific Antibodies

| | Anti-BCMA | Anti-CD3 | |
|---|---|---|---|
| | Antigen-Binding | Antigen-Binding | Common |
| Bispecific | Domain | Domain | Light Chain |
| Antibody | Heavy Chain | Heavy Chain | Variable |
| Identifier | Variable Region | Variable Region | Region |
| REGN5458 | mAb21581 | mAb7221G | mAb7221G |
| REGN5459 | mAb21581 | mAb7221G20 | mAb7221G20 |

Tables 2A and 2B show the amino acid sequence identifiers for the bispecific anti-BCMA x anti-CD3 antibodies exemplified herein.

TABLE 2A
Amino Acid Sequences of Anti-BCMA × Anti-CD3 Bispecific Antibodies (HCVRs)

| Bispecific | Anti-BCMA | | | | Anti-CD3 | | | |
|---|---|---|---|---|---|---|---|---|
| Antibody | First Antigen-Binding Domain | | | | Second Antigen-Binding Domain | | | |
| Identifier | HCVR | HCDR1 | HCDR2 | HCDR3 | HCVR | HCDR1 | HCDR2 | HCDR3 |
| REGN5458 | 1 | 2 | 3 | 4 | 9 | 10 | 11 | 12 |
| REGN5459 | 1 | 2 | 3 | 4 | 13 | 14 | 15 | 16 |

TABLE 2B
Amino Acid Sequences of Anti-BCMA ×
Anti-CD3 Bispecific Antibodies (LCVR)

| | |
|---|---|
| Bispecific | Common Light Chain |
| Antibody | Variable Region |

| Identifier | LCVR | LCDR1 | LCDR2 | LCDR3 |
|---|---|---|---|---|
| REGN5458 | 5 | 6 | 7 | 8 |
| REGN5459 | 5 | 6 | 7 | 8 |

The REGN5458 bispecific antibody identified in Tables 2A and 2B comprises a first heavy chain (containing the first antigen-binding domain) comprising the amino acid sequence of SEQ ID NO: 29, a second heavy chain (containing the second antigen-binding domain) comprising the amino acid sequence of SEQ ID NO: 30, and a common light chain comprising the amino acid sequence of SEQ ID NO: 32. The first heavy chain of the REGN5458 bispecific antibody comprises a constant region comprising the amino acid sequence of SEQ ID NO: 33. The second heavy chain of the REGN5458 bispecific antibody comprises a constant region comprising the amino acid sequence of SEQ ID NO: 34. The common light chain of the REGN5458 bispecific antibody comprises a constant region comprising the amino acid sequence of SEQ ID NO: 35.

The REGN5459 bispecific antibody identified in Tables 2A and 2B comprises a first heavy chain (containing the first antigen-binding domain) comprising the amino acid sequence of SEQ ID NO: 29, a second heavy chain (containing the second antigen-binding domain) comprising the amino acid sequence of SEQ ID NO: 31, and a common light chain comprising the amino acid sequence of SEQ ID NO: 32. The first heavy chain of the REGN5459 bispecific antibody comprises a constant region comprising the amino acid sequence of SEQ ID NO: 33. The second heavy chain of the REGN5459 bispecific antibody comprises a constant region comprising the amino acid sequence of SEQ ID NO: 34. The common light chain of the REGN5459 bispecific antibody comprises a constant region comprising the amino acid sequence of SEQ ID NO: 35.

### Example 2: Desensitization of Chronic Kidney Disease Patients in Need of Kidney Transplantation Who are Highly Sensitized to Human Leukocyte Antigen with Anti-BCMA x Anti-CD3 Bispecific Antibodies

A phase ½ study of bispecific anti-BCMA x anti-CD3 antibodies for desensitization of chronic kidney disease patients in need of kidney transplantation who are highly sensitized to human leukocyte antigen (HLA).

Objectives: Both primary and secondary endpoints will be explored.

The primary obiectives of the study are to assess the safety and tolerability of REGN5459 (Part A) or REGN5458 (Part B) as monotherapy in patients with chronic kidney disease (CKD) who need kidney transplantation and are highly sensitized to human leukocyte antigen (HLA).

The secondary objectives of the study are to determine/assess the following for REGN5459 (Part A) or REGN5458 (Part B): (i) dose regimen(s) that result in a clinically meaningful reduction of anti-HLA alloantibody levels; (ii) effect on calculated panel-reactive antibody (cPRA) levels; (iii) time to maximal and clinically meaningful reduction in anti-HLA alloantibody levels; (iv) duration of the effect of study drug on the reduction of anti-HLA alloantibodies; (v) effect on circulating immunoglobulin (Ig) classes (isotypes); (vi) pharmacokinetic (PK) properties; and (vii) immunogenicity.

Study Design: This is a phase ½ study of REGN5459 (Part A) or REGN5458 (Part B) in patients with chronic kidney disease (CKD) who are on hemodialysis and highly sensitized to HLA. For both Parts A and B, a 3+3 dose escalation design will be used to determine the dose level with acceptable safety, PK, and pharmacologic effect. Part A will open first, and Part B of the study may be opened based on emerging safety data from Part A of this study.

Each patient will receive either REGN5459 (Part A) or REGN5458 (Part B), but not both, at the assigned dose level for 1 cycle of 3 infusions during a 15-day period, as shown for Part A in Table 3 or Part B in Table 4. Patients will be premedicated with 40 mg dexamethasone intravenous (IV) infusion at least 1 hour before each study drug infusion commences to limit the potential for cytokine release syndrome (CRS). If any infusion is not adequately tolerated, doses for subsequent infusions may be modified for that patient and for patients enrolled in additional cohorts.

The planned duration of the study for each patient will be up to approximately 30 weeks, including a screening period (up to 14 days), dosing period (15 days), safety observation period (28 days, starting at day 1), and a follow-up period (approximately 26 weeks, or until patient crossmatches with a potential transplant donor). Patients who successfully crossmatch with a potential transplant donor will discontinue this study and be offered enrollment to a separate protocol (Example 3) for additional assessments after kidney transplantation.

Safety will be assessed by the incidence and severity of treatment-emergent adverse events (TEAEs), graded by the National Cancer Institute Common Terminology Criteria for Adverse Events, version 5.0 (NCI-CTCAE v5.0) except for CRS that will be graded according to Table 5. Activity of drug will be assessed by anti-HLA alloantibody mean fluorescence intensity (MFI) by single antigen bead (SAB) assay. Pharmacokinetic, anti-drug antibody (ADA), and pharmacodynamic (PD) measures will be analyzed from serum/plasma and peripheral blood mononuclear cells before REGN5459 or REGN5458 infusion, and at several time points thereafter. A minimum of 3 patients will be enrolled in each cohort. Additional patients (up to a total of 6 evaluable patients) may be enrolled in any given cohort to further explore safety, PK, and PD characteristics.

Modifications to the dosing schema for Part A and Part B (e.g., lesser dose increments between cohorts and/or between lower and higher doses within cohorts) may be explored based on data observed in previous cohorts.

TABLE 3
Dose Escalation Schema for Part A (REGN5459)

| | | Week 1 (Day 1) | | Week 2 (Day 8) | | Week 3 (Day 15) | |
|---|---|---|---|---|---|---|---|
| Cohort | N | (Initial dose) | | (Intermediate dose) | | (Full dose) | |
| −1* | 3-6 | 0.05 | mg | 0.15 | mg | 0.5 | mg |
| 1 | 3-6 | 0.15 | mg | 0.5 | mg | 1.5 | mg |
| 2 | 3-6 | 0.5 | mg | 1.5 | mg | 5 | mg |
| 3 | 3-6 | 1.5 | mg | 5 | mg | 15 | mg |
| 4 | 3-6 | 5 | mg | 15 | mg | 50 | mg |
| 5 | 3-6 | 5 | mg | 25 | mg | 150 | mg |

*Cohort −1 may be evaluated if cohort 1 is determined to be intolerable.

TABLE 4
Dose Escalation Schema for Part B (REGN5458)

| | | | | Week 2 | | | |
|---|---|---|---|---|---|---|---|
| | | Week 1 | | (Day 8) | | Week 3 | |
| | | (Day 1) | | (Intermediate | | (Day 15) | |
| Cohort [symbol] | N | (Initial dose) | | dose) | | (Full dose) | |
| −1* | 3-6 | 0.05 | mg | 0.15 | mg | 0.5 | mg |
| 1 | 3-6 | 0.15 | mg | 0.5 | mg | 1.5 | mg |
| 2 | 3-6 | 0.5 | mg | 1.5 | mg | 5 | mg |
| 3 | 3-6 | 1 | mg | 3 | mg | 10 | mg |
| 4 | 3-6 | 1 | mg | 5 | mg | 20 | mg |
| 5 | 3-6 | 1 | mg | 5 | mg | 40 | mg |

*Cohort −1 may be evaluated if cohort 1 is determined to be intolerable.

Dose escalation will proceed for Part A and for Part B until an acceptable dose is determined, or maximal dose level is reached, based on the evaluation of safety and any other data available at that time. The maximum full dose that will be evaluated in this study is 150 mg of REGN5459 or 40 mg of REGN5458. However, in the 14-day safety observation period that follows the full dose, if there is the occurrence of an adverse event of interest (AEI) in 1 patient or an adverse event (AE) of grade ≥2 (with the exception of a grade 2 AE that is clearly unrelated to study drug) in 2 or more patients in a dose cohort, but the dose regimen is determined nonetheless to be tolerable, then escalation of the full dose will be no more than 2-fold (i.e., 100% increase) over the full dose from the previously evaluated dose cohort.

An acute IRR is defined as any AE that occurs less than 6 hours from the start of the infusion, or within 2 hours after completion of the infusion (whichever is later), and is associated with typical signs and symptoms including, but not limited to: flushing, tachycardia, hypotension, dyspnea, bronchospasm, back pain, fever, urticaria, edema, nausea, and rashes. Cytokine release syndrome is a disorder characterized by fever, tachypnea, headache, tachycardia, hypotension, rash, and/or hypoxia; in this study it is defined as such an event that occurs 6 or more hours from the start of the infusion, or more than 2 hours after completion of the infusion, whichever is later. Cytokine release syndrome grading and management guidelines, including clinical evaluation and monitoring, are detailed in Table 5.

TABLE 5
Cytokine Release Syndrome Toxicity Grading*

| CRS | | | | |
|---|---|---|---|---|
| Parameter | Grade 1 | Grade 2 | Grade 3 | Grade 4 |
| Fever¹ | Tempera- | Tempera- | Tempera- | Tempera- |
| | ture ≥38° C. | ture ≥38° C. | ture ≥38° C. | ture ≥38° C. |

With

| | | | | |
|---|---|---|---|---|
| Hypotension | None | Not | Requiring | Requiring |
| | | requiring | vasopressors | multiple |
| | | vasopressors | with or | vasopressors |
| | | | without | (excluding |
| | | | vasopressin | vasopressin) |

And/or²

| | | | | |
|---|---|---|---|---|
| Hypoxia | None | Requiring | Requiring | Requiring |
| | | low-flow | high-flow | positive |
| | | nasal | nasal | pressure (eg |
| | | cannula³ or | cannula³, | CPAP, |
| | | blow-by | facemask, | BiPAP, |
| | | | nonrebreather | intubation |
| | | | mask, or | and |
| | | | Venturi mask | mechanical |
| | | | or blow-by | ventilation) |

BiPAP = bilevel positive airway pressure,
CPAP = continuous positive airway pressure,
CRS = Cytokine Release Syndrome
*Adapted from American Society for Transplantation and Cellular Therapy (ASTCT) Cytokine Release Syndrome Consensus Grading.
Organ toxicities associated with CRS may be graded according to CTCAE v5.0, but they do not influence CRS grading.
¹Fever is defined as temperature ≥38° C. not attributable to any other cause. In patients who have CRS and then receive antipyretic or anticytokine therapy such as tocilizumab or steroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
³Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

Study Duration: The planned duration of the study for each patient will be up to approximately 30 weeks, including a screening period (up to 14 days), dosing period (15 days), safety observation period (28 days, starting at day 1), and a follow-up period (approximately 26 weeks, or until patient crossmatches with a potential transplant donor). Patients who successfully crossmatch with a potential transplant donor will discontinue this study and be offered enrollment to a separate protocol (Example 3) for additional assessments after kidney transplantation.

Study Population: There will be approximately 6 to 60 evaluable patients for this study; 6 to 30 for each of Part A and Part B.

The study population will consist of adult patients aged 18 through 70 years with CKD requiring hemodialysis and awaiting kidney transplant on the United Network for Organ Sharing (UNOS) list with a cPRA 99.9%, or those with a cPRA>98% (98.1% to 99.8%) who have spent 5 years or longer on the waitlist.

Inclusion Criteria—A patient must meet the following criteria at screening to be eligible for inclusion in the study:
1. Ages 18 through 70
2. Has CKD requiring hemodialysis, and awaiting kidney transplant on the UNOS list, with a cPRA≥99.9%, or those with a cPRA>98% (98.1% to 99.8%) who have spent 5 years or longer on the waitlist
3. Adequate hematologic function as measured by: Platelet count >50×10⁹/L. A patient may not have received a platelet transfusion within 7 days to meet this platelet eligibility requirement; Absolute neutrophil count (ANC)>1.0×10⁹/L; Hemoglobin >8.0 g/dL
4. Adequate hepatic function, defined as: Total bilirubin ≤1.5×ULN*; Transaminase (ALT, AST)≤2.5×ULN; Alkaline phosphatase ≤2.5×ULN (upper limit of normal)−*Patients with documented history of Gilbert syndrome do not need to meet this total bilirubin requirement provided that the total bilirubin is unchanged from the baseline value
5. Willing and able to comply with clinic visits and study-related procedures
6. Provide informed consent signed by study patient or legally acceptable representative

Exclusion Criteria—A patient who meets any of the following criteria, or has any other medical condition that might preclude the safe administration of study treatment, will be excluded from the study:
1. Current or active malignancy not in remission for at least 1 year
2. CNS pathology or history of CNS neurodegenerative or movement disorders
3. Patients who have had their spleen removed, including patients with functional asplenia
4. Patients who have received a stem cell transplantation within 5 years
5. Body mass index ≥35 kg/m² at screening
6. Hypogammaglobulinemia, defined as total plasma IgG<300 mg/dL at screening
7. Continuous systemic corticosteroid treatment with more than 10 mg per day of prednisone (or anti-inflammatory equivalent) within 72 hours of start of study drug administration
8. Received a calcineurin inhibitor (eg, tacrolimus, cyclosporine) within 30 days of study drug administration
9. Received cyclophosphamide, rituximab, obinutuzumab, other anti-CD20 or B cell-depleting agents, or proteasome inhibitors or anti-CD38 therapies (eg, isatuximab, daratumumab) within 6 months of study drug administration
10. Prior treatment with any anti-BCMA antibody (including antibody drug conjugate or bsAb) or BCMA-directed CAR-T cell therapy
11. Use of investigational agents within 8 weeks or 5 half-lives of study drug administration (whichever is longer)
12. Evidence of active infection on screening chest X-ray with appropriate clinical evidence of infection (Patients may be rescreened after a full treatment course for, and resolution of, active infection)
13. Any infection requiring hospitalization or treatment with IV anti-infectives within 4 weeks of study drug administration
14. Human immunodeficiency virus (HIV)-positive patients at screening
15. Incompletely treated latent or active tuberculosis infection diagnosed by skin testing, interferon-γ release assay, and/or chest imaging within the past 5 years, according to local practice guidelines or Center for Disease Control guidelines. Unclear cases should be discussed with the medical/study director before enrollment.
16. Patients with a history of chronic hepatitis B, and those who test positive for hepatitis B surface antigen (HBsAg) or hepatitis B core antibody (HBcAb) at screening
17. Patients with a history of hepatitis C, or who are hepatitis C virus (HCV) antibody-positive, are excluded. However, patients who are HCV antibody-positive and successfully completed a prior course of anti-HCV therapy, and who have an undetectable HCV ribonucleic acid (RNA) by polymerase chain reaction (PCR), are permitted.
18. Vaccination within 28 days before first study drug administration with a vector that has replicative potential
19. Has received a COVID-19 vaccination within 1 week of planned start of study drug, or for which the planned COVID-19 vaccination would not be completed 1 week before start of study drug
20. Recent recipients of any licensed or investigational live/attenuated vaccines within 2 months of the screening visit, including but not limited to: Adenovirus (Adenovirus vaccine live oral type 7); Varicella-zoster virus (VZV; Varivax®, Zostavax®); Rotavirus vaccine (RotaShield®); Yellow fever vaccine (YF-Vax®); Measles and mumps vaccine (measles and mumps virus vaccine live); Measles, mumps, and rubella vaccine (M-M-R® II); BCG vaccine; Sabin oral polio vaccine; Rabies vaccines (Imovax®, RabAvert®); FluMist® vaccine
21. History of documented severe allergic reactions or acute hypersensitivity reaction attributed to prior IVIG, anticytokine therapy (eg, tocilizumab), study-required vaccines, or premedication treatment
22. Known hypersensitivity to any component of the formulated product
23. Patients who are committed to an institution due to an order issued either by the judicial or the administrative authorities at screening
24. Members of the clinical site study team and/or his/her immediate family, unless prior approval granted by the sponsor
25. Pregnant or breastfeeding women at screening
26. Women of childbearing potential and men who are unwilling to practice highly effective contraception prior to the initial dose/start of the first treatment, during the study, and for at least 4 months after the last dose. Highly effective contraceptive measures for women include: Stable use of combined (estrogen- and progestogen-containing) hormonal contraception (oral, intravaginal, transdermal) or progestogen-only hormonal contraception (oral, injectable, implantable) associated with inhibition of ovulation initiated 2 or more menstrual cycles prior to screening; Intrauterine device (IUD); intrauterine hormone-releasing system (IUS); Bilateral tubal ligation; Vasectomized partner (provided that the male vasectomized partner is the sole sexual partner of the study participant and that the partner has obtained medical assessment of surgical success for the procedure); and/or sexual abstinence.
27. Has any medical condition, comorbidity, physical examination finding, or metabolic dysfunction, or clinical laboratory abnormality that, in the opinion of the investigator renders the patient unsuitable for participation in a clinical study due to high safety risks and/or potential to affect interpretation of results of the study
28. Cardiac ejection fraction <40% by echocardiogram or multi-gated acquisition scan (MUGA
29. Significant cardiovascular disease (eg, New York Heart Association Class Ill or IV cardiac disease, myocardial infarction, stroke, or transient ischemic attack within the previous 6 months, unstable arrhythmias or unstable angina) and/or significant pulmonary disease (eg, obstructive pulmonary disease and history of symptomatic bronchospasm)
30. Documented history of autonomic dysfunction (eg, in Type 1 diabetes) that may preclude the patient's ability to tolerate protocol therapy

Treatment(s): No placebo will be used in this study. The dose administered will be a fixed dose and will not be determined by patient weight or body surface area. Study drug will be administered as an IV infusion.

REGN5459 or REGN5458 (B cell maturation antigen [BCMA]x cluster of differentiation 3 [CD3] bsAbs) for IV infusion (1 cycle of 3 infusions during a 15-day period) will be supplied as a liquid in sterile, single-use vials. Each vial will contain REGN5459 or REGN5458 at a concentration of 10 mg/mL. Diluent for REGN5459 or REGN5458 is supplied as a sterile liquid solution in a glass vial for IV administration.

Dexamethasone 40 mg IV; antihistamine (diphenhydramine [H-1 blocker]) 25 mg IV or PO; acetaminophen (paracetamol) 650 mg PO. Patients should receive premedication with dexamethasone before REGN5459 or REGN5458 administration according to Table 6.

TABLE 6
Premedication with antihistamine,
and acetaminophen (paracetamol)

| Premedication | Dose | Administration |
|---|---|---|
| Dexamethasone | 40 mg IV | At least 1 hr before but not more |
| | | than 3 hr prior to dose |
| Antihistamine | 25 mg IV | 30 to 60 min prior to dose |
| (diphenhydramine [H1- | or PO | |
| blocker]) | | |
| Acetaminophen | 650 mg PO | 30 to 60 min prior to dose |
| (Paracetamol) | | |

### Study Endpoint(s):

The primary endpoints in the study are:
Incidence of AEI from the first study drug dose through end of the safety observation period
Incidence and severity of TEAEs, including AESI and serious adverse events (SAEs), from the first study drug dose up to end of study

The secondary endpoints in the study are:
Proportion of patients with a clinically meaningful reduction in anti-HLA alloantibodies during the study, defined as either: Reduction in cPRA from baseline; or Reduction in the peak (immunodominant) anti-HLA mean fluorescence intensity (MFI) to <5,000, or by >50% (by single antigen bead [SAB] assay)
Maximum reduction in the peak (immunodominant) MFI from baseline
Percent change from baseline in the peak (immunodominant) MFI
Percent change from baseline in the sum of MFI of anti-HLA alloantibodies using the SAB assay
Time to first clinically meaningful reductions in anti-HLA alloantibody levels (defined as peak anti-HLA alloantibody MFI<5,000 or ≥50% reduction) by SAB assay
Time to maximal reduction in anti-HLA alloantibody levels (defined as peak anti-HLA alloantibody MFI<5,000 or ≥50% reduction) by SAB assay
Maximum reduction in cPRA from baseline
Time to first clinically meaningful reduction in cPRA
Time to maximal reduction in cPRA from baseline
Duration of a reduction in peak anti-HLA alloantibody to MFI<5,000 or by ≥50%, and the duration of maximal reductions in anti-HLA alloantibody MFI and % cPRA by SAB assay
Serum concentrations of Ig classes (IgG, IgA, IgE, IgM, IgG1, IgG2, and IgG3) over time
Percent change from baseline of serum concentrations of Ig classes (IgG, IgA, IgE, IgM, IgG1, IgG2, and IgG3) over time
Concentrations of REGN5459 or REGN5458 in serum
Incidence of treatment-emergent antidrug antibodies to REGN5459 or REGN5458 over time.

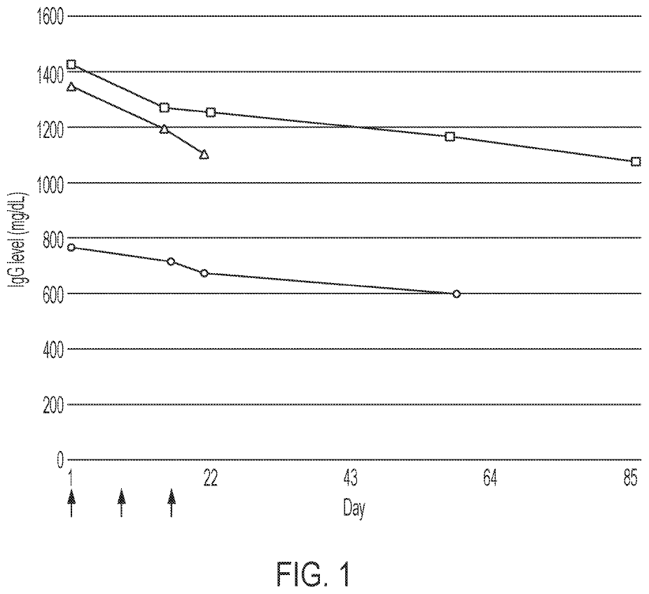

Preliminary Results: A total of 7 highly sensitized participants with chronic kidney disease requiring hemodialysis have been dosed with REGN5459, at full doses from 1.5 mg to 5 mg. All participants (squares, triangles, circles in FIG. 1) enrolled in the 5 mg cohort showed reduction in total serum IgG concentration from baseline (FIG. 1).

### Example 3: Evaluation of Kidney Transplant Recipients Previously Desensitized with Anti-BCMA x Anti-CD3 Bispecific Antibodies

A noninterventional extension study for patients treated in the study discussed in Example 2 with REGN5459 or REGN5458 (BCMA x CD3 Bispecific Antibodies) who receive a kidney transplant.

Objectives: Both primary and secondary endpoints will be explored.

The primary objective of the study is to assess adverse events (AEs) and serious adverse events (SAEs) in kidney transplant recipients previously treated with REGN5459 or REGN5458 in the study discussed in Example 2.

The secondary obiectives of the study are to evaluate each of the following in kidney transplant recipients previously treated with REGN5459 or REGN5458:
Rates and classification of antibody-mediated and T-cell-mediated kidney allograft rejection
Graft survival
Allograft function
Delayed allograft function
Anti-human leukocyte antigen (HLA) alloantibody levels and calculated panel-reactive antibody (cPRA)
Emergence of de novo donor-specific antibodies
Circulating immunoglobulin (Ig) classes (isotypes)
Pharmacokinetics (PK) of REGN5459 or REGN5458

Study Design: All patients treated with REGN5459 or REGN5458 in the study discussed in Example 2 who receive a kidney transplant can be enrolled in this study and will be followed as 2 cohorts/treatment groups (patients who previously received REGN5459 and those who received REGN5458) for AEs, clinical outcomes, and biomarkers. No further study drug will be administered.

Study Duration: This study will continue until the last patient completes 12 months of follow-up assessments post-transplant, or until all patients have discontinued from the study.

Study Population: Six to twelve patients are expected to be enrolled in this study to collect safety and outcomes data in patients aged 18 through 70 years who receive a kidney transplant and were administered REGN5459 or REGN5458 in the study discussed in Example 2.

Inclusion Criteria—A patient must meet the following criteria to be eligible for inclusion in the study:
1. Received at least 1 dose of treatment with REGN5459 or REGN5458 in the study discussed in Example 2
2. Received, or scheduled to receive after acceptable crossmatching, a kidney transplant while enrolled in the study discussed in Example 2
3. Willing and able to comply with clinic visits and study-related procedures
4. Provide informed consent signed by study patient or legally acceptable representative

Exclusion Criteria—There are no exclusion criteria for this study.

### Study Endpoint(s):

The primary endpoints in the study is the incidence of AEs and SAEs over a 12-month period, starting at the time of kidney transplantation.

The secondary endpoints are in the study are to assess the following post-transplant:
Incidence, time to diagnosis, and responsiveness to therapy by 12 months, according to Banff classification, of each of the following types of biopsy-proven kidney allograft rejection:
   Active antibody-mediated rejection (AMR) (Banff classification, Category 2)
   Chronic active AMR (Banff classification, Category 2)
   Acute T-cell-mediated rejection (TCMR) (Banff classification, Category 4)
   Chronic active TCMR (Banff classification, Category 4)
Incidence and timing of graft loss (defined as becoming dialysis-dependent) by 12 months
Change in estimated glomerular filtration rate (eGFR) at 1, 2, 3, 6, and 12 months
Incidence of delayed graft function (defined as the use of dialysis within 7 days post-transplant)
Change (% and mean fluorescence intensity [MFI]) in anti-HLA alloantibodies (using single antigen bead [SAB] assay) compared with pretransplant levels at 2, 3, 6, and 12 months, and at the time of suspected clinical episodes of allograft rejection
Change in cPRA at 1, 2, 3, 6, and 12 months
Change (% and MFI) in donor-specific anti-HLA alloantibodies (using SAB assay and donor HLA type) compared with recipient pretransplant anti-HLA alloantibody levels at 1, 2, 3, 6, and 12 months, and at the time of suspected clinical episodes of allograft rejection
Cumulative incidence of de novo anti-HLA alloantibody development by SAB assay by 12 months
Circulating serum concentrations of Ig classes (IgG, IgA, and IgM) over time
Percent change from baseline of circulating serum concentrations of Ig classes (IgG, IgA, and IgM) over time
Concentrations of REGN5459 or REGN5458 in serum up to 12 months

Results: The incidence of AEs and SAEs following kidney transplantation will be comparable to that of a kidney transplant patient population not having received REGN5459 or REGN5458, and graft loss and graft function will be comparable to that of a kidney transplant patient population not having received REGN5459 or REGN5458.

### Example 4: REGN5459 Causes Depletion of Plasma Cells and Reduces Serum Ig Levels in Humanized Mice

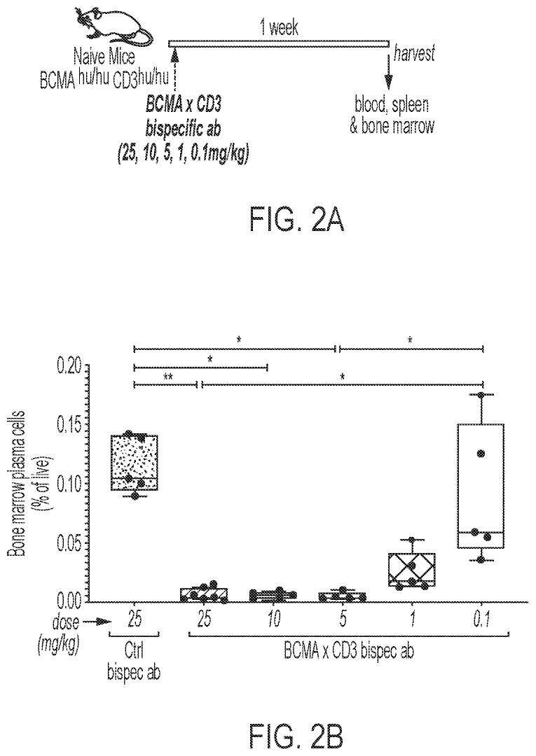

This Example shows T cell-mediated killing of plasma cells (PCs) and decreases in serum Ig levels by a human BCMAxCD3 bispecific antibody (REGN5459) that binds BCMA on PCs via the targeting arm and CD3 on T cells via the effector arm. The activity of REGN5458 was evaluated in genetically humanized BCMAhu/hu/CD3hu/hu mice (U.S. Pat. No. 11,384,153) (FIG. 2A).

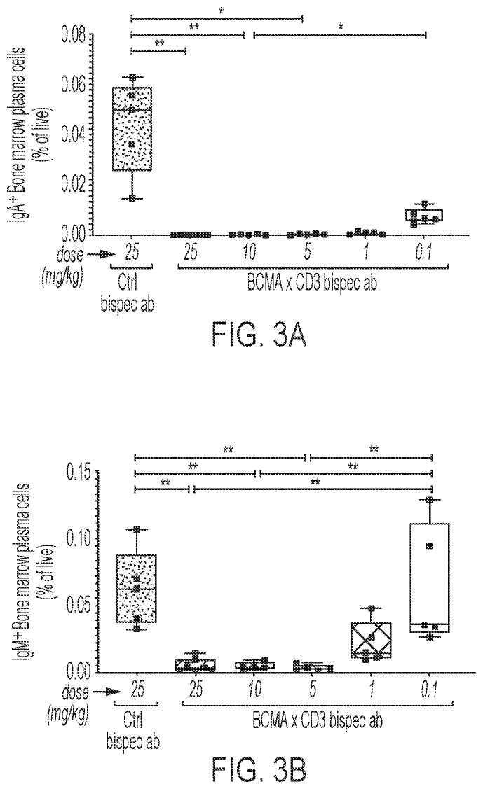

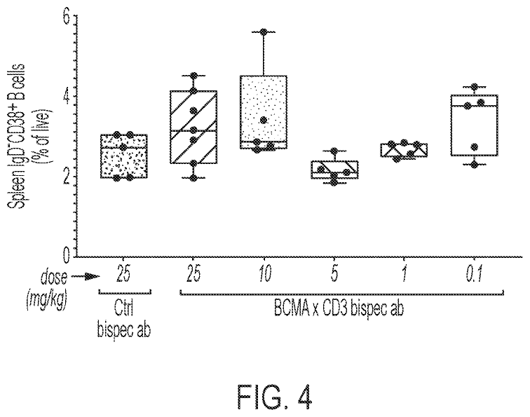

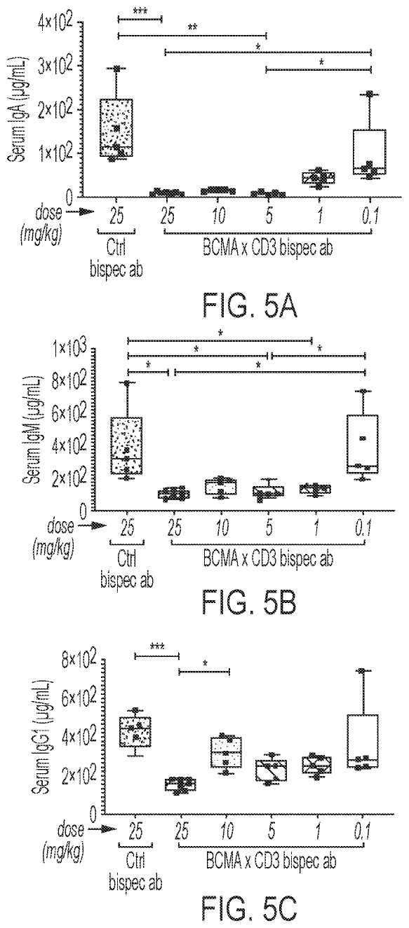

BCMAxCD3 bispecific antibody administration resulted in robust and statistically significant reduction in bone marrow plasma cells (BMPCs) that was comparable at all doses between 5 and 25 mg/kg and was equivalent across different isotype-specific PC subsets (FIG. 2B, FIGS. 3A and 3B). Importantly, splenic B cells, including antigen-experienced CD38+IgD-follicular B cells, were not significantly impacted by BCMAxCD3 bispecific antibody (FIG. 4). The plasma cell depletion was reflected by significant decreases of serum IgA, IgM and IgG1 levels, the magnitude of which match their expected half-lives. (FIGS. 5A, 5B and 5C). This finding is consistent with IgA having the shortest half-life in circulation relative to IgM and IgG1, which allows pre-existing IgA to decrease ˜10-20-fold within one week of elimination of its sources.

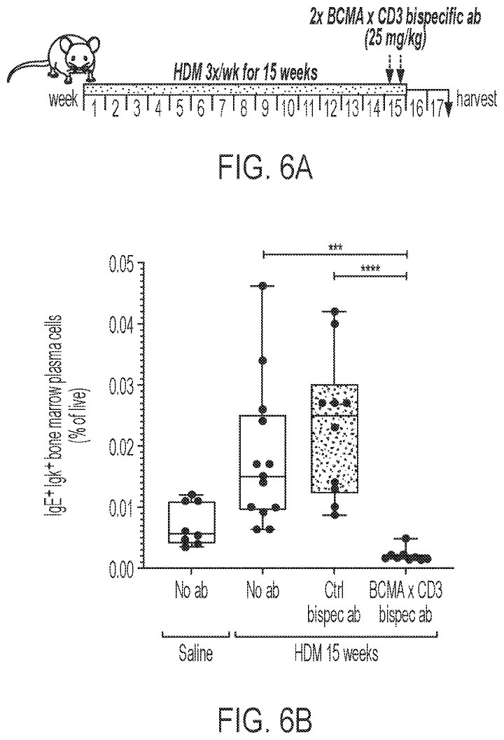

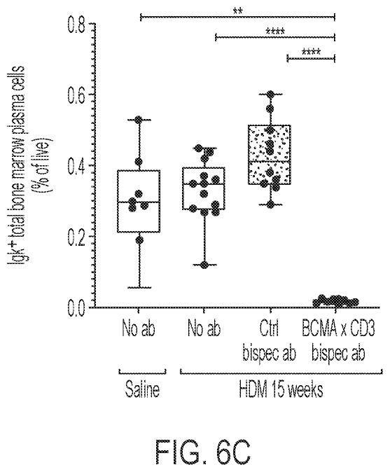

In a second experiment, the impact of the BCMAxCD3 bispecific antibody on antigen-specific Ig was evaluated in a 15-week model of intranasal house dust mite (HDM) exposure (Asrat et al 2020, Sci Immunol 5) (FIG. 6A). Overall BMPCs, including IgE+BMPCs, were nearly eliminated within two weeks of administration of the bispecific antibody (FIGS. 6B and 6C). Total circulating IgA and IgM and IgG1 levels were assessed and were found to be transiently decreased after BCMAxCD3 bispecific antibody treatment.

Thus, BCMA x CD3 bispecific antibody efficiently depletes (transiently) plasma cells including IgE plasma cells, but not antigen-experienced B cells in BCMAhu/huCD3hu/hu mice.

### Example 5: Effect of a BCMAxCD3 Bispecific Antibody on Plasma Cells and Circulating Antibody Levels in Monkeys

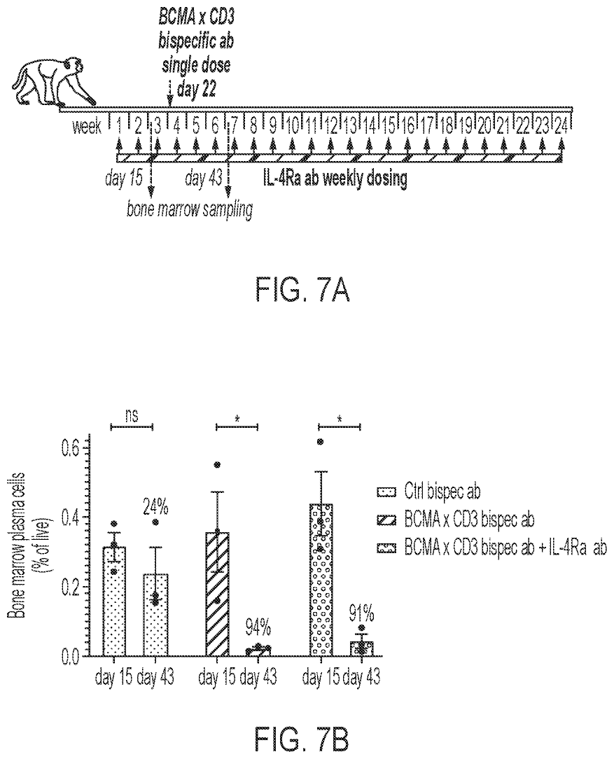

The effect of a BCMAxCD3 bispecific antibody (which is known to also bind monkey BCMA (Haber et al 2021, Sci Rep 11, 14397), alone or in combination with a monkey IL-4Ra antibody (REGN646) was evaluated in cynomolgus monkeys (FIG. 7A). A single dose of the BCMAxCD3 bispecific antibody resulted in a rapid and robust decrease in BMPCs (FIG. 7B). Consistent with the observations in mice (in Example 4), this decrease in BMPCs was closely mirrored by rapidly decreasing IgE and IgA levels, which reached the nadir 5 weeks after BCMAxCD3 bispecific antibody administration. Remarkably, following the nadir the IgA levels gradually recovered in animals treated with BCMAxCD3 bispecific antibody, both alone and in combination with anti-IL-4Ra, whereas IgE levels recovered in animals treated with BCMAxCD3 bispecific antibody alone, but not in those treated in combination with anti-IL-4Ra.

### Example 6: Impact of BCMAxCD3 Bispecific Antibody on Human Serum Immunoglobulins

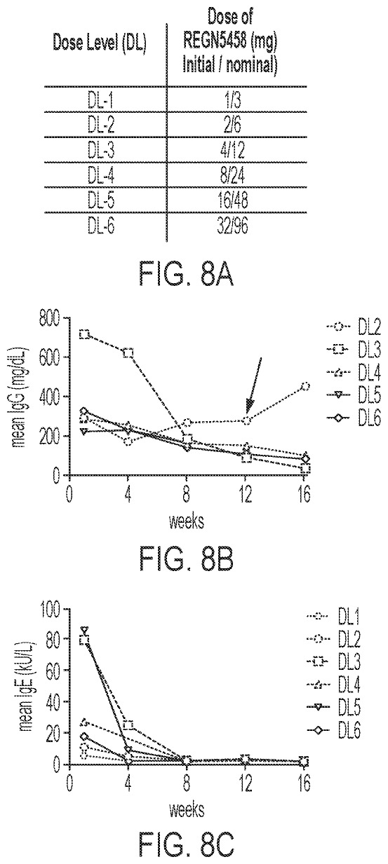

The effect of weekly administration of a BCMAxCD3 bispecific antibody (REGN5458) was evaluated in patients with multiple myeloma (FIG. 8A). REGN5458 resulted in robust reduction in immunoglobulin levels over time, with IgE levels completely reduced as early as 4 weeks post treatment (FIG. 8C). IgG levels were also depleted by more than 50% between 4-8 weeks consistent with the half-life in humans (FIG. 8B).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 7
Sequence Excluded from ST.26-Formatted Sequence Listing

| SEQ ID NO: | Sequence |
|---|---|
| 7 | AAS |

What is claimed is:

1. A method of reducing an alloantibody level in a subject in need of a solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell.

2. The method of claim 1, wherein reducing the alloantibody level in the subject comprises reducing the alloantibody level below a baseline alloantibody level measured prior to administration of the bispecific antibody or antigen-binding fragment thereof.

3. The method of claim 1 or 2, further comprising measuring a baseline alloantibody level in the subject prior to administration of the bispecific antibody or antigen-binding fragment thereof.

4. The method of claim 2 or 3, wherein the alloantibody level and/or the baseline alloantibody level is measured by a single antigen bead assay, and the alloantibody level and/or the baseline alloantibody level corresponds to a peak immunodominant anti-HLA antibody mean fluorescence intensity.

5. The method of claim 4, wherein the reduction in the alloantibody level corresponds to a reduction in a peak immunodominant anti-HLA antibody mean fluorescence intensity by ≥50% relative to the baseline alloantibody level.

6. The method of claim 1, wherein the alloantibody level is measured by a single antigen bead assay, and a reduction in the alloantibody level corresponds to a reduction in a peak immunodominant anti-HLA antibody mean fluorescence intensity to <5000.

7. A method of reducing a calculated panel reactive antibody (cPRA) level of a subject in need of a solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell.

8. The method of claim 7, wherein reducing the cPRA level of the subject comprises reducing the cPRA level of the subject below a baseline cPRA level determined prior to administration of the bispecific antibody or antigen-binding fragment thereof.

9. The method of claim 7 or 8, wherein the cPRA level of the subject:
(a) is reduced to <99%;
(b) is reduced to <98%;
(c) is reduced to <97%;
(d) is reduced to <96%;
(e) is reduced to <95%;
(f) is reduced to <94%;
(g) is reduced to <93%;
(h) is reduced to <92%;
(i) is reduced to <91%;
(j) is reduced to <90%;
(k) is reduced to <89%;
(l) is reduced to <88%;
(m) is reduced to <87%;
(n) is reduced to <86%;
(o) is reduced to <85%;
(p) is reduced to <84%;
(q) is reduced to <83%;
(r) is reduced to <82%;
(s) is reduced to <81%; or
(t) is reduced to <80%.

10. A method of reducing sensitization of a subject to anti-HLA antibodies prior to solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell, wherein the subject's risk of rejection at the time of organ transplant is no greater than that of a control population with a pre-transplant calculated panel reactive antibody (cPRA) level of 90%.

11. The method of claim 10, wherein the subject's risk of rejection at the time of organ transplant is no greater than that of a control population with a pre-transplant cPRA level of 80%.

12. The method of claim 11, wherein the subject's risk of rejection at the time of organ transplant is no greater than that of a control population with a pre-transplant cPRA level of from 50% to <80%.

13. A method of reducing risk of allograft rejection in a subject following a solid organ transplant, the method comprising administering to the subject a bispecific antibody or antigen-binding fragment thereof comprising a first antigen-binding domain that specifically binds a human B-cell maturation antigen (BCMA) on a plasma cell, and a second antigen-binding domain that specifically binds human CD3 on a T cell, wherein the subject's risk of rejection during a post-transplant interval is no greater than that of a control population with a pre-transplant calculated panel reactive antibody (cPRA) level of 90%.

14. The method of claim 13, wherein the subject's risk of rejection during the post-transplant interval is no greater than that of a control population with a pre-transplant cPRA level of 80%.

15. The method of claim 14, wherein the subject's risk of rejection during the post-transplant interval is no greater than that of a control population with a pre-transplant cPRA level of from 50% to <80%.

16. The method of any one of claims 13-15, wherein the post-transplant interval is a 3-month interval beginning one day after organ transplant.

17. The method of any one of claims 13-15, wherein the post-transplant interval is a 6-month interval beginning one day after organ transplant.

18. The method of any one of claims 13-15, wherein the post-transplant interval is a 12-month interval beginning one day after organ transplant.

19. The method of any one of claims 13-18, wherein graft function is maintained in the subject during the post-transplant interval at a function level equal to or greater than that of the control population.

20. The method of any one of claims 1-19, wherein the subject is a human.

21. The method of any one of claims 1-20, wherein the solid organ is selected from the group consisting of a kidney, a lung, a pancreas, or a heart.

22. The method of claim 21, wherein the solid organ is a kidney.

23. The method of any one of claims 1-22, wherein the subject has chronic kidney disease and requires hemodialysis.

24. The method of any one of claims 1-23, wherein the first antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 1; and
(b) three light chain complementarity determining regions (LCDR1, LCDR2 and LCDR3) contained within a light chain variable region (LCVR) comprising the amino acid sequence of SEQ ID NO: 5.

25. The method of claim 24, wherein the first antigen-binding domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4.

26. The method of claim 24 or 25, wherein the first antigen-binding domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

27. The method of claim 24, wherein the first antigen-binding domain comprises a HCVR comprising the amino acid sequence of SEQ ID NO: 1, and a LCVR comprising the amino acid sequence of SEQ ID NO: 5.

28. The method of any one of claims 1-27, wherein the second antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 9 or SEQ ID NO: 13; and
(b) three light chain complementarity determining regions (LCDR1, LCDR2 and LCDR3) contained within a light chain variable region (LCVR) comprising the amino acid sequence of SEQ ID NO: 5.

29. The method of claim 28, wherein the second antigen-binding domain comprises:
(a) a HCDR1 comprising the amino acid sequence of SEQ ID NO: 10 or SEQ ID NO: 14;
(b) a HCDR2 comprising the amino acid sequence of SEQ ID NO: 11 or SEQ ID NO: 15; and
(c) a HCDR3 comprising the amino acid sequence of SEQ ID NO: 12 or SEQ ID NO: 16.

30. The method of claim 28 or 29, wherein the second antigen-binding domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

31. The method of claim 30, wherein the second antigen-binding domain comprises:
(a) HCDR1, HCDR2, HCDR3 domains, respectively, comprising the amino acid sequences of SEQ ID NOs: 10, 11, 12; and LCDR1, LCDR2, LCDR3 domains, respectively, comprising the amino acid sequences of SEQ ID NOs: 6, 7, 8; or
(b) HCDR1, HCDR2, HCDR3 domains, respectively, comprising the amino acid sequences of SEQ ID NOs: 14, 15, 16; and LCDR1, LCDR2, LCDR3 domains, respectively, comprising the amino acid sequences of SEQ ID NOs: 6, 7, 8.

32. The method of claim 31, wherein the second antigen-binding domain comprises:
(a) a HCVR comprising the amino acid sequence of SEQ ID NO: 9, and a LCVR comprising the amino acid sequence of SEQ ID NO: 5; or
(b) a HCVR comprising the amino acid sequence of SEQ ID NO: 13, and a LCVR comprising the amino acid sequence of SEQ ID NO: 5.

33. The method of any one of claims 1-23, wherein:
(a) the first antigen-binding domain comprises HCDR1, HCDR2, HCDR3 domains, respectively, consisting of the amino acid sequences of SEQ ID NOs: 2, 3, 4, and LCDR1, LCDR2, LCDR3 domains, respectively, consisting of the amino acid sequences of SEQ ID NOs: 6, 7, 8; and
(b) the second antigen binding domain comprises HCDR1, HCDR2, HCDR3 domains, respectively, consisting of the amino acid sequences of SEQ ID NOs: 10, 11, 12, and LCDR1, LCDR2, LCDR3 domains, respectively, consisting of the amino acid sequences of SEQ ID NOs: 6, 7, 8.

34. The method of any one of claims 1-23, wherein:
(a) the first antigen-binding domain comprises HCDR1, HCDR2, HCDR3 domains, respectively, consisting of the amino acid sequences of SEQ ID NOs: 2, 3, 4, and LCDR1,LCDR2,LCDR3 domains, respectively, consisting of the amino acid sequences of SEQ ID NOs: 6, 7, 8; and
(b) the second antigen binding domain comprises HCDR1,HCDR2,HCDR3 domains, respectively, consisting of the amino acid sequences of SEQ ID NOs: 14, 15, 16, and LCDR1,LCDR2,LCDR3 domains, respectively, consisting of the amino acid sequences of SEQ ID NOs: 6, 7, 8.

35. The method of claim 33, wherein:
(a) the first antigen binding domain comprises a HCVR consisting of the amino acid sequence of SEQ ID NO: 1, and a LCVR consisting of the amino acid sequence of SEQ ID NO: 5; and
(b) the second antigen binding domain comprises a HCVR consisting of the amino acid sequence of SEQ ID NO: 9, and a LCVR consisting of the amino acid sequence of SEQ ID NO: 5.

36. The method of claim 34, wherein:
(a) the first antigen binding domain comprises a HCVR consisting of the amino acid sequence of SEQ ID NO: 1, and a LCVR consisting of the amino acid sequence of SEQ ID NO: 5; and
(b) the second antigen binding domain comprises a HCVR consisting of the amino acid sequence of SEQ ID NO: 13, and a LCVR consisting of the amino acid sequence of SEQ ID NO: 5.

37. The method of any one of claims 1-36, wherein the bispecific antibody or antigen-binding fragment thereof is a bispecific antibody comprising a human IgG heavy chain constant region.

38. The method of claim 37, wherein the bispecific antibody comprises a heavy chain comprising a constant region comprising the amino acid sequence of SEQ ID NO: 33.

39. The method of claim 37 or 38, wherein the bispecific antibody comprises a heavy chain comprising a constant region comprising the amino acid sequence of SEQ ID NO: 34.

40. The method of claim 37, wherein the human IgG heavy chain constant region is isotype IgG1.

41. The method of claim 37, wherein the human IgG heavy chain constant region is isotype IgG4.

42. The method of claim 40 or 41, wherein the bispecific antibody comprises a chimeric hinge that reduces Fcγ receptor binding relative to a wild-type hinge of the same isotype.

43. The method of any one of claims 1-36, wherein the bispecific antibody or antigen-binding fragment thereof is a bispecific antibody comprising a first heavy chain comprising the amino acid sequence of SEQ ID NO: 29, a second heavy chain comprising the amino acid sequence of SEQ ID NO: 30, and a common light chain comprising the amino acid sequence of SEQ ID NO: 32.

44. The method of any one of claims 1-36, wherein the bispecific antibody or antigen-binding fragment thereof is a bispecific antibody comprising a first heavy chain comprising the amino acid sequence of SEQ ID NO: 29, a second heavy chain comprising the amino acid sequence of SEQ ID NO: 31, and a common light chain comprising the amino acid sequence of SEQ ID NO: 32.

45. The method of any one of claims 1-44, wherein the bispecific antibody is administered in a dosing regimen comprising a split initial dose.

46. The method of any one of claims 1-45, wherein the bispecific antibody is administered to the subject at a dose of from 0.05 mg to 150 mg weekly.

47. A dosing regimen for use in the method of any one of claims 1-46, wherein the dosing regimen comprises administration of a bispecific antibody to the subject at an initial dose during week one of the dosing regimen, at a secondary dose during week two of the dosing regimen, and at a tertiary dose during week three of the dosing regimen, wherein the tertiary dose is equal to or greater than the secondary dose, and the secondary dose is greater than the initial dose.

48. The dosing regimen of claim 47, wherein the initial dose is from 0.05 mg to 5 mg.

49. The dosing regimen of claim 47 or 48, wherein the secondary dose is from 0.15 mg to 25 mg.

50. The dosing regimen of any one of claims 47-49, wherein the tertiary dose is from 0.5 mg to 150 mg.

51. The dosing regimen of any one of claims 47-50, wherein the initial dose is 0.05 mg, 0.15 mg, 0.5 mg, 1 mg, 1.5 mg or 5 mg.

52. The dosing regimen of any one of claims 47-51, wherein the secondary dose is 0.15 mg, 0.5 mg, 1.5 mg, 3 mg, 5 mg, 15 mg or 25 mg.

53. The dosing regimen of any one of claims 47-52, wherein the tertiary dose is 0.5 mg, 1.5 mg, 5 mg, 10 mg, 15 mg, 20 mg, 40 mg, 50 mg or 150 mg.


SEQUENCE LISTING (35 sequences)

SEQ ID NO: 1 - protein, 125 aa, synthetic construct
  REGION 1..125: note = Synthetic
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 2 - protein, 8 aa, synthetic construct
  REGION 1..8: note = Synthetic
GFTFSNFW

SEQ ID NO: 3 - protein, 8 aa, synthetic construct
  REGION 1..8: note = Synthetic
MNQDGSEK

SEQ ID NO: 4 - protein, 18 aa, synthetic construct
  REGION 1..18: note = Synthetic
ARDREYCISTSCYDDFDY

SEQ ID NO: 5 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 6 - protein, 6 aa, synthetic construct
  REGION 1..6: note = Synthetic
QSISSY

SEQ ID NO: 7 - skipped

SEQ ID NO: 8 - protein, 10 aa, synthetic construct
  REGION 1..10: note = Synthetic
QQSYSTPPIT

SEQ ID NO: 9 - protein, 124 aa, synthetic construct
  REGION 1..124: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 10 - protein, 8 aa, synthetic construct
  REGION 1..8: note = Synthetic
GFTFDDYS

SEQ ID NO: 11 - protein, 8 aa, synthetic construct
  REGION 1..8: note = Synthetic
ISWNSGSK

SEQ ID NO: 12 - protein, 17 aa, synthetic construct
  REGION 1..17: note = Synthetic
AKYGSGYGKFYHYGLDV

SEQ ID NO: 13 - protein, 124 aa, synthetic construct
  REGION 1..124: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 14 - protein, 8 aa, synthetic construct
  REGION 1..8: note = Synthetic
GFTFDDYS

SEQ ID NO: 15 - protein, 8 aa, synthetic construct
  REGION 1..8: note = Synthetic
ISWNSGSI

SEQ ID NO: 16 - protein, 17 aa, synthetic construct
  REGION 1..17: note = Synthetic
AKYGSGYGKFYYYGMDV

SEQ ID NO: 17 - protein, 82 aa, synthetic construct
  REGION 1..82: note = Synthetic
MLQMAGQCSQNEYFDSLLHACIPCQLRCSSNTPPLTCQRYCNASVTNSVKGTNAEQKLISEEDLGGEQKLISEEDLHHHHHH

SEQ ID NO: 18 - protein, 287 aa, synthetic construct
  REGION 1..287: note = Synthetic
MLQMAGQCSQNEYFDSLLHACIPCQLRCSSNTPPLTCQRYCNASVTNSVKGTNAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 19 - protein, 81 aa, synthetic construct
  REGION 1..81: note = Synthetic
MLQMARQCSQNEYFDSLLHDCKPCQLRCSSTPPLTCQRYCNASMTNSVKGMNAEQKLISEEDLGGEQKLISEEDLHHHHHH

SEQ ID NO: 20 - protein, 286 aa, synthetic construct
  REGION 1..286: note = Synthetic
MLQMARQCSQNEYFDSLLHDCKPCQLRCSSTPPLTCQRYCNASMTNSVKGMNAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 282 aa, synthetic construct
  REGION 1..282: note = Synthetic
MAQQCFHSEYFDSLLHACKPCHLRCSNPPATCQPYCDPSVTSSVKGTYTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 184 aa, synthetic construct
  REGION 1..184: note = Synthetic
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 23 - protein, 207 aa, synthetic construct
  REGION 1..207: note = Synthetic
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 24 - protein, 171 aa, synthetic construct
  REGION 1..171: note = Synthetic
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 25 - protein, 164 aa, synthetic construct
  REGION 1..164: note = Synthetic
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 182 aa, synthetic construct
  REGION 1..182: note = Synthetic
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 27 - protein, 281 aa, synthetic construct
  REGION 1..281: note = Synthetic
MLQMAGQCSQNEYFDSLLHACIPCQLRCSSNTPPLTCQRYCNASVTNSVKGTNADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 43 aa, synthetic construct
  REGION 1..43: note = Synthetic
MLQMAGQCSQNEYFDSLLHACIPCQLRCSSNTPPLTCQRYCNA

SEQ ID NO: 29 - protein, 451 aa, synthetic construct
  REGION 1..451: note = Synthetic
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 450 aa, synthetic construct
  REGION 1..450: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 31 - protein, 450 aa, synthetic construct
  REGION 1..450: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 32 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 326 aa, synthetic construct
  REGION 1..326: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 35 - protein, 107 aa, synthetic construct [sequence omitted]